(19) United States
(12) Patent Application Publication
(10) Pub. No.: US 2024/0317788 A1
(43) Pub. Date: Sep. 26, 2024

(54) PRECIOUS METAL COMPLEXES WITH DIHYDROAZULENYL LIGANDS AND USE THEREOF

(71) Applicant: UMICORE AG & CO. KG, Hanau-Wolfgang (DE)

(72) Inventors: Tobias VOLLGRAFF, Marburg (DE); Joerg SUNDERMEYER, Marburg (DE); Angelino DOPPIU, Seligenstadt (DE)

(73) Assignee: UMICORE AG & CO. KG, Hanau-Wolfgang (DE)

(21) Appl. No.: 18/262,532

(22) PCT Filed: Feb. 4, 2022
(86) PCT No.: PCT/EP2022/052719

§ 371 Date: Jul. 21, 2023

(30) Foreign Application Priority Data
Feb. 18, 2021 (EP) 21158012.1
Jun. 1, 2021 (EP) 21177066.4

(51) Int. Cl. C07F 17/02; B01J 31/22; C07C 13/52; C07F 7/08; C08G 77/08; C23C 16/18; C23C 16/455

(52) U.S. Cl. CPC C07F 17/02; B01J 31/2295; C07C 13/52; C07F 7/0879; C08G 77/08; C23C 16/18; C23C 16/45525; C07C 2602/30

(57) ABSTRACT

The invention relates to a method for producing complexes of precious metals, in particular platinum, which have at least one organo-dihydroazulenyl ligand. The invention also relates to complexes of precious metals, in particular platinum, which have at least one organo-dihydroazulenyl ligand and to the use of the aforementioned metal complexes as precatalysts or catalysts in a chemical reaction or as precursor compounds for producing a layer which contains a precious metal, in particular platinum, or a metal layer consisting of a precious metal, in particular platinum, in particular on at least one surface of a substrate. The invention additionally relates to a substrate, in particular a substrate which can be obtained according to such a method. The invention also relates to a crosslinkable silicon composition comprising at least one compound with aliphatic carbon-carbon multi-bonds, at least one compound with Si-bonded hydrogen atoms, and at least one platinum (IV) complex of the aforementioned type. The invention also relates to novel alkali metal organo-dihydroazulenyls which can be used to produce metal complexes, in particular of the aforementioned type.

Homoleptic and heteroleptic metal complexes having anionic ligands derived from bicyclo[5.3.0]decapentaene (azulene) or singly or multiply substituted azulene, i.e., azulene derivatives, and methods for their preparation are known.

Azulene and azulene derivatives, which, for example, have an alkyl or aryl substituent instead of one H atom at the 4 position, belong to the ortho-fused aromatic ring systems. They are present as neutral zwitterions.

Anionic ligands derived from mineral oil-based azulene or typically mineral oil-based azulene derivatives can be prepared, for example, by adding an alkali metal organyl, for example methyllithium or phenyllithium, in the 4 position, in the 6 position or in the 8 position of an azulene molecule or azulene derivative. The respective product is an alkali metal dihydroazulenide that carries an organyl group, for example, a methyl group or a phenyl group, in addition to an H atom in the 4 position, 6 position or 8 position. Therefore, there is an RCH group in the C4 position or in the C6 position or in the C8 position of the azulene skeleton, wherein R is an organyl group.

It should be noted that azulene in the 4 position, 6 position and 8 position has a comparable electrophilicity. Therefore, the formation of regioisomers can occur. Because azulene also has C₂ᵥ symmetry, the products of the addition reaction are an alkali metal 4-organo-dihydroazulenide and/or an alkali metal 6-organo-dihydroazulenide. The anion in question can be referred to as an 4-organo-dihydroazulenyl anion or as an 6-organo-dihydroazulenyl anion. More specifically, it is a 4-organo-3a,4-dihydroazulenyl anion or a 6-organo-3a,6-dihydroazulenyl anion.

Regardless of the substitution pattern, the aromaticity is limited to the five-membered ring by the alkyl or aryl addition. As a rule, the blue color that is typical of azulene is lost.

The aforementioned organo-dihydroazulenyl anions, for example an organo-dihydroguaiazulenyl anion derived from the natural substance 7-iso-propyl-1,4-dimethylazulene (=guaiazulene), are cyclopentadienyl-like monoanions or derivatives of the cyclopentadienyl anion (Cp⁻). Therefore, like the cyclopentadienyl anion, they are suitable as ligands for the preparation of metallocene type sandwich complexes. Lithium dihydroazulenides have some advantages over lithium cyclopentadienide (LiCp), in particular in terms of preparation and storage.

A disadvantage of the chemistry of the cyclopentadienyl ligand, one of the most important ligands of metal organics, is that it is oil-based. The provision of LiCp first involves the thermal cracking of dicyclopentadiene into its monomer cyclopentadiene in the presence of a catalyst, for example iron powder. After this first step, cyclopentadiene must be used quickly or stored in the freezer. Otherwise, it rapidly dimerizes back to dicyclopentadiene. In a second step, cyclopentadiene is reacted with a strong base, for example a lithium alkyl, usually the relatively expensive n-butyllithium.

Examples of transition metal complexes that have dihydroazulenyl anions, i.e., cyclopentadienyl-like monoanions or cyclopentadienyl derivatives as ligands, are described inter alia in a review by M. R. Churchill. (Prog. Inorg. Chem. 1970, 54-98, Chapter IV, Section C.)

Hafner and Weldes (Liebigs Ann. Chem. 1957, 606, 90-99) investigated the behavior of azulene with respect to organometallic compounds. Starting from methyllithium and azulene (equimolar), they obtained the dietherate of lithium-4-methyl-dihydroazulene. The reaction proceeded exothermically. In addition, butyllithium, phenyllithium, and sodium and potassium organic compounds were reacted with azulene and the corresponding azulenes substituted in the 4 position were obtained.

The group working with Edelmann (J. Richter, P. Liebing, F. T. Edelmann, Inorg. Chim. Acta 2018, 475, 18-27) only recently published their results on the synthesis of metallocene complexes of the early transition metals titanium and zirconium and the lanthanide neodymium, which have dihydroazulenyl ligands. The lithium dihydroazulenides used in the context of the metal complex syntheses, namely lithium-4-methyl dihydroazulenide, lithium-7-iso-propyl-1,4,8-trimethyl-dihydroazulenide and lithium-7-iso-propyl-1,4-dimethyl-8-phenyl-dihydroazulenide, were prepared analogously to the method of Hafner and Weldes (Liebigs Ann. Chem. 1957, 606, 90-99), i.e., starting from azulene or guaiazulene and methyl or phenyllithium. However, in order to obtain solvent-free products, the isolated lithium dihydroazulenides were reacted with n-pentane instead of diethyl ether.

The authors summarize that dihydroazulenyl anions and similar anions derived from azulene as ligands for the preparation of metallocene of early transition metals and the lanthanoids initially seemed promising to them. However, according to the synthesis protocols described by them, the metallocene complexes can in most cases only be obtained as isomer mixtures. The synthetic value of dihydroazulenyl ligands for organometallic chemistry or mono- and bis-dihydroazulenyl complexes derived therefrom can therefore be significantly reduced.

On the one hand, the complex compounds prepared by Edelmann's group have the disadvantage that they are mixed zirconocenes, namely zirconocene dichlorides. Due to the presence of chloride ligands, the possibilities of further use of these metal complexes can be restricted. On the other hand, the complexes described by Edelmann and his collaborators are obtained only in low yields of 19% to 48%. It is also disadvantageous that the plurality of metal complexes, in particular those prepared using guaiazulene, have relatively high melting points, namely above 150° C., in some cases even above 200° C. This is disadvantageous with regard to a possible use of the complexes based on guaiazulene, for example as precursors in a vapor deposition process, in particular a low-temperature vapor deposition process.

Overall, the synthesis routes known from the literature for the production of metal complexes having organo-dihydroazulenyl ligands are to be classified as unsatisfactory from a technological, ecological and (atom) economical perspective. Consequently, this also applies to the complex compounds producible by means of the previously known methods. In addition, metal complexes of precious metals, such as platinum, which are relevant for a wide range of applications and have at least one organo-dihydroazulenyl ligand, have not yet been provided.

It is therefore an object of the invention to overcome these and further disadvantages of the prior art and to provide a method for preparing complexes of precious metals, in particular platinum, that have at least one organo-dihydroazulenyl ligand. This method should make it possible to prepare metal complexes of the aforementioned type in high purity and good yield in a simple, reproducible and comparatively inexpensive manner. The method should also be characterized by the fact that it can also be carried out on an industrial scale with a comparable yield and purity of the target compounds. In addition, complexes of precious metals, in particular platinum, that have at least one organo-dihydroazulenyl ligand, are to be provided. The present invention also relates to the use of such complexes. Novel alkali metal organo-dihydroazulenyls that can be used to prepare metal complexes, in particular of the aforementioned type, are also to be provided.

The main features of the invention are defined in the claims.

The object is achieved by a compound according to general formula

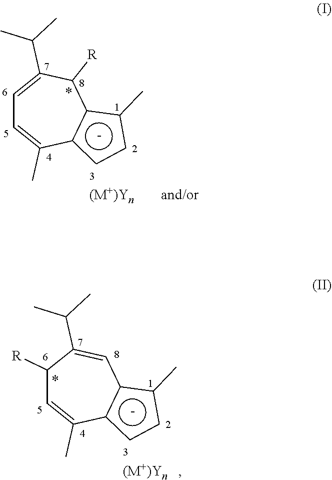

wherein
   M⁺ is an alkali metal cation,
   R is selected from the group consisting of primary, secondary, tertiary alkyl, alkenyl and alkynyl radicals having 1 to 10 carbon atoms, cyclic alkyl radicals having 3 to 10 carbon atoms, a benzyl radical, mononuclear aryl radicals, polynuclear aryl radicals, mononuclear heteroaryl radicals and polynuclear heteroaryl radicals,
   Y is a neutral ligand that is bound or coordinated to M⁺ via at least one donor atom, wherein H₂O is excluded, and
   n=0, 1, 2, 3 or 4,
wherein
the compounds lithium-7-iso-propyl-1,4,8-trimethyl-dihydroazulenide and lithium-7-iso-propyl-1,4-dimethyl-8-phenyl-dihydroazulenide and their THF adducts and DME adducts are excluded.

7-iso-propyl-1,4-dimethylazulene (=guaiazulene, hereafter abbreviated to Gua) is a natural substance that contains chamomile oil and other essential oils and is therefore inexpensive to obtain in large quantities. It can be produced synthetically from the guaiacol of guaiac wood oil (guaiac resin). Guaiazulene is an intensely blue substance with anti-inflammatory properties.

THF stands for tetrahydrofuran, and DME is the abbreviation for 1,2-dimethoxyethane.

In the case of the compound according to the formula I, the carbon atom C8 is a stereocenter. In the case of the compound according to formula II, the carbon atom C6 is a stereocenter. In the two structural formulae shown above and in some selected structural formulae shown in the following, the stereocenters are marked with an asterisk (δ).

The compounds claimed herein are referred to in the present case as alkali metal dihydroguaiazulenides, alkali metal-R-dihydroguaiazulenides or alkali metal organo-dihydroguaiazulenides, unless a particular regioisomer is meant, i.e., an alkali metal-8-R-dihydroguaiazulenide (formula I) or an alkali metal-6-R-dihydroguaiazulenide (formula II). The claimed alkali metal-dihydroguaiazulenides may be isomerically pure or be a mixture of the two regioisomers according to formula I and II.

The compounds according to the general formulae I and II each have an organo-dihydroazulenyl anion or R-dihydroguaiazulenyl anion (GuaR)¹⁻, which carries an organyl radical R in the 8 position or in the 6 position of the guaiazulene skeleton in addition to an H atom. The R-dihydroguaiazulenyl anion (GuaR)¹⁻ can therefore be a 7-iso-propyl-1,4-dimethyl-8-R-dihydroazulenyl anion or 8-R-dihydroguaiazulenyl anion (Gua-8-R)¹⁻ according to formula I or a 7-iso-propyl-1,4-dimethyl-6-R-dihydroazulenyl anion or 6-R-dihydroguaiazulenyl anion (Gua-6-R)¹⁻ according to formula II. In other words: There is an RCH group in the C8 position or in the C6 position of the guaiazulene skeleton. As a result of the addition of an organyl anion (R)¹⁻, the aromaticity is limited to the five-membered ring, wherein the blue color typical for azulene and derivatives thereof is generally lost. The organo-dihydroazulenyl ligand anion (GuaR)¹⁻ is a derivative of the cyclopentadienyl anion or a cyclopentadienyl-like monoanion.

The two excluded compounds are each an alkali metal dihydroguaiazulenide according to formula I, wherein M⁺ is in each case Li⁺ and R=methyl or phenyl. These compounds, the THF solvates thereof and the DME adduct of lithium-7-iso-propyl-1,4,8-trimethyl-dihydroazulenide were described by Edelmann and his collaborators (see J. Richter, P. Liebing, F. T. Edelmann Inorg. Chim. Acta 2018, 475, 18-27) and, just like the DME solvates of the two excluded compounds, are not the subject matter of the present invention. Mixtures of the two aforementioned compounds or of the THF solvates of these compounds or of the DME adduct of lithium-7-iso-propyl-1,4,8-trimethyl-dihydroazulenide having the respective regioisomer according to formula II were not described by the authors.

The claimed compounds can be adduct-free or solvent-free, in particular when n=0, or can be present as solvent adducts or solvates having one (n=1), two (n=2), three (n=3) or four (n=4) neutral ligands Y In a solution, solvation of the alkali metal ions M⁺ takes place regularly, in particular when the solvent used is an alkoxyalkane or comprises at least one alkoxyalkane. In a solution, solvated lithium ions are in particular present, wherein Y=one alkoxyalkane and n=4, for example Li(OC₂H₅)₄ or Li(thf)₄. In addition, isolated compounds according to formulae I and II can be present as solvent adducts, wherein the respective alkali metal cation M⁺ is complexed, for example by a crown ether.

According to the present invention, the term “alkoxyalkane” means any oxygen-containing ether, for example also glycol dialkyl ethers and crown ethers. The glycol dialkyl ethers are also understood to mean terminally dialkylated mono-, di- or trialkylene glycol dialkyl ethers. Crown ethers are macrocyclic polyethers in which the ring is composed of a plurality of ethyloxy units (—CH₂—CH₂—O—). They are capable of complexing cations to form crown ethers. When the inner diameter of a crown ether and an ion radius of a metal cation match, extremely stable complexes are formed. For example, the crown ether [18]-crown-6 is a very good ligand for a potassium ion, while, for example, the crown ether [15]-crown-5 is particularly well suited for complexing a sodium ion. For example, a lithium ion can be very well complexed by the crown ether [12]-crown-4. By exchanging the oxygen atoms with other heteroatoms, for example nitrogen, phosphorus or sulfur, aza-, phospha- or thia-derivatives of the crown ethers are accessible.

Whether the compounds according to the general formula I or II claimed here are present as solvent adducts, in particular as alkoxyalkane adducts, depends on various factors, for example on the solvent used for the preparation of compounds according to the general formula I or II and also on the type of alkali metal cation M⁺. For example, if a solvent comprising or consisting of one or more alkoxyalkanes was used in the course of the synthesis, including purification, or an alkoxyalkane was used for crystallization, for example a crown ether, and at least one alkylation or arylation reagent having lithium cations was used, a solvent adduct Li(alkoxyalkane)˜(GuaR), wherein n=1 or 2, will most likely be present. If such a solvent adduct is finally washed with a non-ethereal solvent, such as pentane or hexane, a solvent-free compound according to the general formula Li(GuaR) is usually present.

The radical R may also be primary, secondary, tertiary alkyl, alkenyl and alkynyl radicals having 2, 3, 4, 5, 6, 7, 8 or 9 carbon atoms, and cyclic alkyl radicals having 4, 5, 6, 7, 8 or 9 carbon atoms.

It is particularly advantageous that the compounds claimed herein, comprising cyclopentadienyl-like monoanions (GuaR)¹⁻, have good to very good long-term stability at room temperature. No decomposition reactions, oligomerization or polymerization is observed during storage at room temperature for several months. This is particularly advantageous with regard to the further use of the compounds according to the general formulae I and II, in particular as ligand precursors for the preparation of sandwich and semi-sandwich complexes. Furthermore, the preparation of alkali metal dihydroguaiazulenides according to the general formulae I and II, comprising cyclopentadienyl-like monoanions (GuaR)¹⁻, is less labor and time-intensive compared to the provision of LiCp. In particular, the compounds claimed herein, which comprise cyclopentadienyl-like ligands, can be prepared using inexpensive renewable raw materials. Guaiazulene is partially accessible synthetically, namely starting from the natural material guaiacol and other azulene formers by simple dehydration and dehydrogenation (T. Shono, N. Kise, T. Fujimoto, N. Tominaga, H. Morita, J. Org. Chem. 1992, 57, 26, 7175-7187; CH 314 487 A (B. Joos) Jan. 29, 1953).

The compounds of the formulae I and II described herein, comprising cyclopentadienyl-like monoanions (GuaR)¹⁻, are simple and reproducible and, depending on the choice of reactants, can be obtained in a sustainable and comparatively cost-effective manner. In addition, a high purity of 97%, advantageously higher than 97%, in particular higher than 98% or 99%, and good yields of typically 60% and good space-time yields can be realized. They are therefore suitable as ligand precursors for the preparation of sandwich and semi-sandwich complexes, even on an industrial scale. In general, the end product may still contain residues of solvents or for example impurities from the reactants. It is known to a person skilled in the art that the content of impurities, for example solvents, can be determined by gas chromatography (GC) methods, optionally with mass spectrometry coupling (GC-MS).

According to the present invention, “purity” means the absence of unwanted impurities, in particular those caused by reactants, by-products, atmospheric oxygen, water, oxygen-containing compounds, semi-metals, metals and solvents. Purity can be important with respect to the subsequent use of the compounds according to formula I and formula II, in particular as ligand precursors for the preparation of the sandwich and semi-sandwich complexes.

The term “space-time yield” is used here to refer to a product quantity formed in a reaction container or reaction vessel per space and time, i.e., the mass of a product obtained per volume and time. For example, kg/L*h is selected as unit.

The terms “reaction container” and “reaction vessel” in the context of the present invention are used synonymously and are not limited to a volume, material composition, equipment, or form. Suitable reaction vessels include, for example, glass flasks, enameled reactors, stirred tank reactors, pressure vessels, tube reactors, microreactors, and flow reactors.

One embodiment of the compounds claimed herein provides that R is selected from the group consisting of
primary, secondary, tertiary alkyl-, alkenyl- and alkynyl radicals having 1 to 6 carbon atoms,
cyclic alkyl radicals having 3 to 6 carbon atoms,
a benzyl radical,
mononuclear aryl residues and polynuclear aryl residues
and
mononuclear heteroaryl radicals and polynuclear heteroaryl radicals.

The radical R may therefore also be primary, secondary, tertiary alkyl, alkenyl and alkynyl radicals having 2, 3, 4 or 5 carbon atoms, and cyclic alkyl radicals having 4 or 5 carbon atoms.

In another embodiment of the compounds according to the general formula I or II, R is selected from the group consisting of Me, Et, n-Pr, i-Pr, n-Bu, i-Bu, s-Bu, t-Bu, n-pentyl, 2-pentyl, 3-pentyl, 2-methylbutyl, 3-methylbutyl, 3-methylbut-2-yl, 2-methylbut-2-yl, 2,2-dimethylpropyl, cyclopentyl, cyclohexyl, phenyl, tolyl, benzyl and cumyl, and isomers thereof. According to yet another advantageous embodiment, R is selected from the group consisting of Me, Et, n-Pr, i-Pr, n-Bu, i-Bu, s-Bu, t-Bu, cyclohexyl, phenyl, tolyl, benzyl and cumyl, and isomers thereof. Particularly advantageously, R is selected from the group consisting of Me, Et, n-Pr, i-Pr, n-Bu, i-Bu, s-Bu, t-Bu, in particular R=Me.

According to a further embodiment of the compounds claimed herein,
i. there is an isomerically pure compound according to general formula I or general formula II
or
ii. there is an isomer mixture containing a first regioisomer according to formula I and a second regioisomer according to formula II and consisting in particular of the first regioisomer according to formula I and the second regioisomer according to formula II.

While the carbon atoms C4, C6 and C8 of bicyclo[5.3.0]decapentaene (=azulene) are known to have comparable nucleophilicities, the carbon atoms C6 and C8 of guaiazulene have different nucleophilicities, namely due to the asymmetric substitution in the C1, C4 and C7 positions. Thus, the compounds according to formula I and II claimed herein can be present, as a function of the nucleophilically acting organyl anion (R)¹⁻, not only as isomer mixtures containing or consisting of the two regioisomers according to formula I and II, but also as isomerically pure compounds according to either formula I or II.

In the context of the present invention, the term “isomerically pure” means that the desired product is obtained or produced in an isomerically pure manner or that the desired isomer is present in an amount of ≥90%, preferably ≥95%, particularly preferably ≥99% after purification. The isomeric purity is determined, for example, by means of nuclear magnetic resonance spectroscopy, in particular by means of ¹H-NMR spectroscopy.

In the case of an isomerically pure compound, either an alkali metal-8-R-dihydroguaiazulenide (formula I) or an alkali metal-6-R-dihydroguaiazulenide (formula II) is present. In other words: There is only one regioisomer that has an RCH group, and thus a chiral center, either in the C8 position (formula I) or in the C6 position (formula II) of the guaiazulene skeleton.

If an isomer mixture is present which contains the first regioisomer according to formula I and the second regioisomer according to formula II, and in particular consists of the first regioisomer according to formula I and the second regioisomer according to formula II, then a mixture is present that contains an alkali metal-8-R-dihydroguaiazulenide (formula I) and an alkali metal-6-R-dihydroguaiazulenide (formula II), in particular consisting of an alkali metal-8-R-dihydroguaiazulenide (formula I) and an alkali metal-6-R-dihydroguaiazulenide (formula II). In formula I, R is identical to R in formula II. In other words: The first regioisomer has an RCH group in the C8 position (formula I) of the guaiazulene skeleton, and the second regioisomer has an RCH group in the C6 position (formula II) of the guaiazulene skeleton.

Another variant of the compounds claimed herein provides that an isomer ratio of first regioisomer: second regioisomer is ≥80:20 and <90:10, advantageously between 81:19 and 89:11, in particular between 82:18 and 88:12, for example 83:17 or 84:16 or 85:15 or 86:14 or 87:13. The isomer ratio is determined, for example, by means of nuclear magnetic resonance spectroscopy, in particular by means of ¹H-NMR spectroscopy.

According to a further embodiment of the compounds according to formulae I and II claimed herein, the alkali metal cation M⁺ is selected from the group consisting of Li⁺, Na⁺ and K⁺.

According to another advantageous embodiment, there is an isomer mixture consisting of a first regioisomer according to formula I.1 and a second regioisomer according to formula II.1

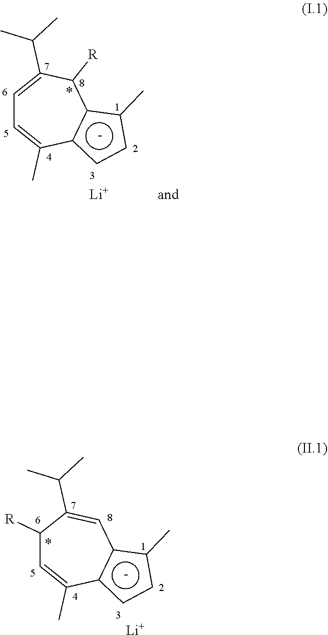

wherein M⁺=Li⁺. R is in particular a methyl group (Me). In an alternative or additional embodiment, the isomer ratio of first regioisomer: second regioisomer is ≥80:20 and <90:10, advantageously between 81:19 and 89:11, in particular between 82:18 and 88:12, for example 83:17 or 84:16 or 85:15 or 86:14 or 87:13. In an alternative or additional embodiment, a number n of the neutral ligand Y is bound or coordinated to M⁺ via at least one donor atom, wherein H₂O is excluded and n=1, 2, 3 or 4. Y can be, for example, an alkoxyalkane and n=4 is possible, and the alkali metal cation, in particular the lithium cation, can be present as a solvate, for example, Li(OC₂H₅)₄ or Li(thf)₄, or as a solvent adduct, for example in Li(OC₂H₅)₂ (GuaR).

A further embodiment of the compounds according to formula I and formula II claimed herein provides that the neutral ligand Y is a polar aprotic solvent. The polar aprotic solvent is advantageously selected from the group consisting of alkoxyalkanes, thioethers and tertiary amines, in particular from the group consisting of alkoxyalkanes and thioethers. The respective compound according to formula I or formula II can then be present in crystalline form as a function of the choice of the neutral ligand Y, for example in the case of diethyl ether, THF, thiophene or triethylamine.

The term “alkoxylalkane” has already been defined above. The term “thioether” comprises both non-cyclic and cyclic thioethers.

In another variant of the compounds claimed herein, the neutral ligand Y is a crown ether selected from the group consisting of macrocyclic polyethers and aza-, phospha- and thia-derivatives thereof, wherein an inner diameter of the crown ether and an ion radius of the respective alkali metal cation M⁺ correspond to each other. The respective compound according to formula I or formula II can then be present in crystalline form.

According to a further embodiment of the compounds claimed herein, a glycol dialkyl ether is provided as a neutral ligand Y, selected from the group consisting of a monoethylene glycol dialkyl ether, a diethylene glycol dialkyl ether, a triethylene glycol dialkyl ether, a monopropylene glycol dialkyl ether, a dipropylene glycol dialkyl ether, a tripropylene glycol dialkyl ether, a monooxomethylene dialkyl ether, a dioxomethylene dialkyl ether and a trioxomethylene dialkyl ether, the isomer mixtures thereof, and mixtures thereof.

In a further embodiment of the compounds claimed herein, the glycol dialkyl ether provided as a neutral ligand Y is selected from the group consisting of ethylene glycol dimethyl ether CH₃—O—CH₂CH₂—O—CH₃, ethylene glycol diethyl ether CH₃CH₂—O—CH₂CH₂—O—CH₂CH₃, ethylene glycol di-n-propyl ether CH₃CH₂CH₂—O—CH₂CH₂—O—CH₂CH₂CH₃, ethylene glycol di-iso-propyl ether (CH₃)₂CH—O—CH₂CH₂O—CH(CH₃)₂, ethylene glycol di-n-butyl ether CH₃CH₂CH₂CH₂—O—CH₂CH₂—O—CH₂CH₂CH₂CH₃, ethylene glycol di-n-pentyl ether CH₃CH₂CH₂CH₂CH₂—O—CH₂CH₂—O—CH₂CH₂CH₂CH₂CH₃, ethylene glycol di-n-hexyl ether CH₃CH₂CH₂CH₂CH₂CH₂—O—CH₂CH₂—O—CH₂CH₂CH₂CH₂CH₂CH₃, ethylene glycol diphenyl ether C₆H₅—O—CH₂CH₂—O—C₆H₅, ethylene glycol dibenzyl ether C₆H₅CH₂—O—CH₂CH₂—O—CH₂C₆H₅, diethylene glycol dimethyl ether CH₃—O—CH₂CH₂—O—CH₂CH₂—O—CH₃, diethylene glycol diethyl ether CH₃CH₂—O—CH₂CH₂—O—CH₂CH₂—O—CH₂CH₃, diethylene glycol di-n-propylether CH₃CH₂CH₂—O—CH₂CH₂—O—CH₂CH₂—O—CH₂CH₂CH₃, diethylene glycol di-iso-propylether (CH₃)₂CH—O—CH₂CH₂—O—CH₂CH₂O—CH(CH₃)₂, diethylene glycol di-n-butylether CH₃CH₂CH₂CH₂—O—CH₂CH₂—O—CH₂CH₂—O—CH₂CH₂CH₂CH₃, diethylene glycol di-n-pentylether CH₃CH₂CH₂CH₂CH₂—O—CH₂CH₂—O—CH₂CH₂—O—CH₂CH₂CH₂CH₂CH₃, diethylene glycol di-n-hexylether CH₃CH₂CH₂CH₂CH₂CH₂—O—CH₂CH₂—O—CH₂CH₂—O—CH₂CH₂CH₂CH₂CH₂CH₃, diethylene glycol diphenyl ether C₆H₅—O—CH₂CH₂—O—CH₂CH₂—O—C₆H₅, diethylene glycol dibenzyl ether C₆H₅CH₂—O—CH₂CH₂—O—CH₂CH₂—O—CH₂C₆H₅, propylene glycol dimethyl ether CH₃—O—CH₂CH₂CH₂—O—CH₃, propylene glycol diethyl ether CH₃CH₂—O—CH₂CH₂CH₂—O—CH₂CH₃, propylene glycol di-n-propyl ether CH₃CH₂CH₂—O—CH₂CH₂CH₂—O—CH₂CH₂CH₃, propylene glycol di-n-butyl ether CH₃CH₂CH₂CH₂—O—CH₂CH₂CH₂—O—CH₂CH₂CH₂CH₃, propylene glycol di-n-pentyl ether CH₃CH₂CH₂CH₂CH₂—O—CH₂CH₂CH₂—O—CH₂CH₂CH₂CH₂CH₃, propylene glycol di-n-hexyl ether CH₃CH₂CH₂CH₂CH₂CH₂—O—CH₂CH₂CH₂—O—CH₂CH₂CH₂CH₂CH₂CH₃, propylene glycol diphenyl ether C₆H₅—O—CH₂CH₂CH₂—O—C₆H₅, propylene glycol dibenzyl ether C₆H₅CH₂—O—CH₂CH₂CH₂—O—CH₂C₆H₅, iso-propylene glycol dimethyl ether CH₃—O—CH₂—CH(CH₃)—O—CH₃, iso-propylene glycol diethyl ether CH₃CH₂—O—CH₂—CH(CH₃)—O—CH₂CH₃, iso-propylene glycol di-n-propyl ether CH₃CH₂CH₂—O—CH₂—CH(CH₃)—O—CH₂CH₂CH₃, iso-propylene glycol di-iso-propyl ether (CH₃)₂CH—O—CH₂—CH(CH₃)—O—CH(CH₃)₂, iso-propylene glycol di-n-butyl ether CH₃CH₂CH₂CH₂—O—CH₂—CH(CH₃)—O—CH₂CH₂CH₂CH₃, iso-propylene glycol di-n-pentyl ether CH₃CH₂CH₂CH₂CH₂—O—CH₂—CH(CH₃)—O—CH₂CH₂CH₂CH₂CH₃, iso-propylene glycol di-n-hexyl ether CH₃CH₂CH₂CH₂CH₂CH₂—O—CH₂—CH(CH₃)—O—CH₂CH₂CH₂CH₂CH₂CH₃, iso-propylene glycol diphenyl ether C₆H₅—O—CH₂—CH(CH₃)—O—C₆H₅, iso-propylene glycol dibenzyl ether C₆H₅CH₂—O—CH₂—CH(CH₃)—O—CH₂C₆H₅, dipropylene glycol dimethyl ether CH₃OCH₂CH₂CH₂OCH₂CH₂CH₂OCH₃, di-iso-propylene glycol di-n-propyl ether CH₃CH₂CH₂—O—CH₂CH(CH₃)—OCH₂CH(CH₃)—O—CH₂CH₂CH₃, tripropylene glycol dimethyl ether CH₃OCH₂CH₂CH₂OCH₂CH₂CH₂OCH₂CH₂CH₂OCH₃, dipropylene glycol dibutyl ether CH₃CH₂CH₂CH₂OCH₂CH₂CH₂OCH₂CH₂CH₂OCH₂CH₂CH₂CH₃, tripropylene glycol dibutyl ether CH₃CH₂CH₂CH₂OCH₂CH₂CH₂OCH₂CH₂CH₂OCH₂CH₂CH₂OCH₂CH₂CH₂CH₃, and mixtures thereof. The stated glycol ethers can also be present as isomer mixtures.

A further embodiment of the compounds claimed herein provides that the neutral ligand Y is an ether. For example, the ether can be a non-cyclic or a cyclic ether selected from the group consisting of dialkyl ethers, cyclopentyl methyl ether, tetrahydrofuran, 2-methyltetrahydrofuran, 3-methyltetrahydrofuran, tetrahydropyran, 1,4-dioxane, 1,2-dimethoxyethane, and isomers thereof, and mixtures thereof, in particular from the group consisting of diethyl ether, methyl tert-butyl ether, di-n-propyl ether, di-iso-propyl ether, di-n-butyl ether, di-iso-butyl ether, di-tert-butyl ether, tetrahydrofuran, 2-methyltetrahydrofuran, 3-methyltetrahydrofuran, tetrahydropyran, 1,4-dioxane, 1,2-dimethoxyethane, and isomers thereof, and mixtures thereof.

The object is also achieved by the use of a compound according to general formula

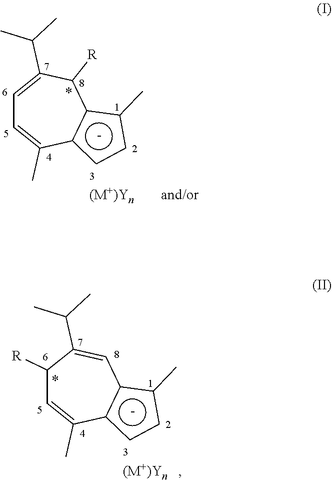

wherein
   M⁺ is an alkali metal cation,
   R is selected from the group consisting of primary, secondary, tertiary alkyl, alkenyl and alkynyl radicals having 1 to 10 carbon atoms, cyclic alkyl radicals having 3 to 10 carbon atoms, a benzyl radical, mononuclear aryl radicals, polynuclear aryl radicals, mononuclear heteroaryl radicals and polynuclear heteroaryl radicals,
   Y is a neutral ligand that is bound or coordinated to M⁺ via at least one donor atom, wherein H₂O is excluded,
and
   n=0, 1, 2, 3 or 4,
or according to one of the embodiments described further above,
for producing a platinum(IV) complex according to the general formula

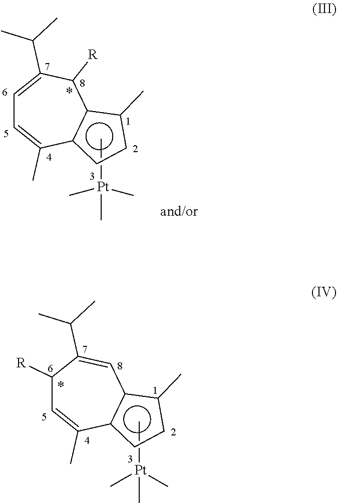

wherein
R is selected from the group consisting of primary, secondary, tertiary alkyl, alkenyl and alkynyl radicals having 1 to 10 carbon atoms, cyclic alkyl radicals having 3 to 10 carbon atoms, a benzyl radical, mononuclear aryl radicals, polynuclear aryl radicals, mononuclear heteroaryl radicals and polynuclear heteroaryl radicals.

The aforementioned use of a compound according to general formula I and/or according to general formula II for producing a platinum(IV) complex according to general formula III and/or IV is a method for producing a platinum(IV) complex according to general formula

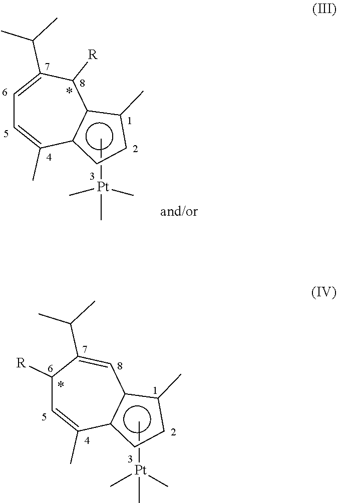

wherein
R is selected from the group consisting of primary, secondary, tertiary alkyl, alkenyl and alkynyl radicals having 1 to 10 carbon atoms, cyclic alkyl radicals having 3 to 10 carbon atoms, a benzyl radical, mononuclear aryl radicals, polynuclear aryl radicals, mononuclear heteroaryl radicals and polynuclear heteroaryl radicals,
using a compound according to general formula

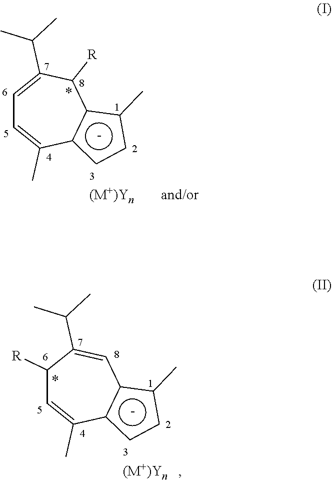

wherein
   M⁺ is an alkali metal cation,
   R is selected from the group consisting of primary, secondary, tertiary alkyl, alkenyl and alkynyl radicals having 1 to 10 carbon atoms, cyclic alkyl radicals having 3 to 10 carbon atoms, a benzyl radical, mononuclear aryl radicals, polynuclear aryl radicals, mononuclear heteroaryl radicals and polynuclear heteroaryl radicals,
   Y is a neutral ligand that is bound or coordinated to M⁺ via at least one donor atom, wherein H₂O is excluded,
and
   n=0, 1, 2, 3 or 4,
or according to one of the embodiments described further above.

The method comprises the following steps:
A. providing
   the compound according to general formula I and/or general formula II
and
   a platinum precursor,
B. synthesizing the platinum(IV) complex according to general formula III and/or general formula IV
using the compound according to general formula I and/or general formula II as a reactant
in a solvent S_{P},
C. optionally isolating the platinum(IV) complex synthesized in step B according to general formula III and/or general formula IV.

In this case, the general formulae III and IV each comprise both the monomers and any oligomers, in particular dimers, and solvent adducts. In addition, the compounds according to general formulae III and IV, which can be produced by means of the claimed method, are in particular each present as a diastereomer mixture. In other words: In the case of the platinum(IV) complex according to formula III, the carbon atom C8 is a stereocenter. In the case of the platinum(IV) complex according to formula IV, the carbon atom C6 is a stereocenter.

Alternatively, a diastereomer mixture comprising, in particular consisting of, the diastereomer mixture of the compound according to formula III and the diastereomer mixture of the compound according to formula IV, can be obtained, in particular as a function of the selected substituent R.

In each of the aforementioned cases, each diastereomer is present as an enantiomer mixture, wherein the diastereomers can be present independently of one another as a racemate.

A person skilled in the art will know which platinum precursors are commercially available or can be prepared—optionally also in situ- and which reaction conditions, for example stoichiometry of the reactants, solvents, reaction temperature, reaction time, and working steps, optionally including necessary solvent exchange, isolation and optionally purification, are required in step B. for the synthesis of the platinum(IV) complex according to formula III and/or formula IV.

The order in which a reaction vessel is loaded with the reactants, i.e., the alkali metal dihydroguaiazulenides according to formula I and/or formula II and a platinum precursor, is freely selectable. This also includes the possibility of carrying out steps A. and B. or sub-steps thereof, i.e. all the steps relating to the preparation of the respective target compound, in a single step, i.e. introducing all reactants and solvents simultaneously or virtually simultaneously into the reaction vessel.

The terms “reaction container” and “reaction vessel” are already defined further above.

The method for producing platinum(IV) complexes according to formulae III and IV described herein can be carried out as a discontinuous process or as a continuous process.

The alkali metal dihydroguaiazulenide used in each case can be present in an isomerically pure form or as a mixture of the two regioisomers according to formulae I and II, i.e., as a mixture comprising an alkali metal 8-R-dihydroguaiazulenide (formula I) and an alkali metal 6-R-dihydroguaiazulenide (formula II) or consisting of an alkali metal 8-R-dihydroguaiazulenide (formula I) and an alkali metal 6-R-dihydroguaiazulenide (formula II). In addition, the compounds used according to formula I and formula II can be present in an adduct-free or solvent-free form, namely when n=0, or as solvent adducts or solvates having one (n=1), two (n=2), three (n=3) or four (n=4) neutral ligands Y A non-limiting selection of neutral ligands Y is given further above.

The solvent S_{P} may also be a mixture of solvents.

Surprisingly, by means of the use of a compound according to general formula I and/or II claimed herein or by means of the method described herein, the aforementioned compounds can be used to obtain platinum(IV) complexes according to general formula III and/or IV, in particular as diastereomer mixtures, in a high purity of 97%, advantageously higher than 97%, in particular higher than 98% or 99%, and in a good yield of typically 60, and in a good space-time yield. In general, the end product may still contain residues of solvents or for example impurities from the reactants. It is known to a person skilled in the art that the content of impurities, for example solvents, can be determined by gas chromatography (GC) methods, optionally with mass spectrometry coupling (GC-MS).

A definition of the term “space-time yield” has already been given above.

A platinum(IV) complex obtainable or obtained in a high purity by means of the method described herein has a total content of impurities, comprising in particular impurities of reactants, by-products, atmospheric oxygen, water, oxygen-containing compounds, semi-metals, metals, for example platinum(0), optionally in the form of platinum(0) nanoparticles and/or nanoparticles containing platinum(0), and solvents, of below 1000 ppm, ideally of below 100 ppm. Purity can be important with regard to the later use of the platinum(IV) complexes that can be prepared by means of this method.

It is surprising, in particular, that strongly reducing organo-dihydroazulenyl ligands (GuaR)¹⁻, namely the 8-R-dihydroguaiazulenyl ligand (Gua-8-R)¹⁻ and the 6-R-dihydroguaiazulenyl ligand (Gua-6-R)¹⁻, are suitable for the preparation of complexes of precious metals, in particular platinum, for example starting from platinum(IV) precursor compounds such as trimethylplatinum(IV) iodide. These complexes, for example, the semi-sandwich complex [PtMe₃(GuaMe)], are additionally advantageously obtainable in a good yield and high purity.

The aforementioned facts are surprising because a person skilled in the art will know that strongly reducing R-dihydroguaiazulenyl ligands (GuaR)¹⁻, such as the 8-R-dihydroguaiazulenyl ligand (Gua-8-R)¹⁻, are ideal in particular for complex formation with early transition metals, for example titanium and zirconium (see J. Richter, P. Liebing, F. T. Edelmann Inorg. Chim. Acta 2018, 475, 18-27).

The compounds of the formulae I and II used as reactants, comprising cyclopentadienyl-like monoanions (GuaR)¹⁻, are simple, reproducible, sustainable and comparatively cost-effective to obtain. In addition, high purity, good yields and space-time yields can be achieved. They are therefore also suitable for use in industrial processes. It is particularly advantageous that the compounds according to formulae I and II used in the context of the method claimed herein, comprising cyclopentadienyl-like monoanions (GuaR)¹⁻, have good to very good long-term stability at room temperature. No decomposition reactions, oligomerization or polymerization is observed during storage at room temperature for several months. Furthermore, the preparation of alkali metal dihydroguaiazulenides according to the general formulae I and II used is less labor and time-intensive compared to the provision of LiCp. In particular, the compounds used as reactants herein, which comprise cyclopentadienyl-like ligands, can be prepared using inexpensive renewable raw materials. This is because guaiazulene is partially accessible synthetically, namely starting from the natural material guaiacol and other azulene formers by simple dehydration and dehydrogenation (T. Shono, N. Kise, T. Fujimoto, N. Tominaga, H. Morita, J. Org. Chem. 1992, 57, 26, 7175-7187; CH 314 487 A (B. Joos) Jan. 29, 1953).

All of the above shows that platinum(IV) complexes are obtainable by means of the method claimed herein, said complexes being an inexpensive and sustainable alternative to previously known catalysts or precatalysts such as [PtMe₃(Cp)] and [PtMe₃(MeCp)]. For example, the guaiazulene required for the preparation of compounds according to formulae III and IV, for example [PtMe₃(GuaMe)], is synthesized using renewable raw materials instead of crude oil. Consequently, the method described herein and the compounds that can be prepared therewith are particularly advantageous, both from an economic and an environmental point of view, compared to the platinum(IV) compounds containing cyclopentadienyl anions mentioned above.

According to one embodiment of the compound claimed herein or the method claimed herein, the solvent S_{P} comprises at least one solvent selected from the group consisting of polar aprotic solvents, aliphatic hydrocarbons, aromatic hydrocarbons, organosilicon compounds, and mixtures thereof. The solvent S_{P} is advantageously selected from the group consisting of polar aprotic solvents, aliphatic hydrocarbons, in particular having 1 to 30 carbon atoms, aromatic hydrocarbons, organosilicon compounds, and mixtures thereof.

The polar aprotic solvent is, for example, an ether or comprises at least one ether. The ether can be selected from the group consisting of tetrahydrofuran, methyl tetrahydrofuran, 1,4-dioxane, 1,2-dimethoxyethane, diethyl ether, methyl tert-butyl ether, di-n-propyl ether, di-iso-propyl ether, cyclopentyl methyl ether, and isomers thereof, and mixtures thereof.

Aliphatic hydrocarbons in the present case are acyclic, cyclic, saturated and unsaturated hydrocarbons. The aliphatic hydrocarbon can also have 2, 3, 4, 5, 6, 7, 8, 9, 10, 11, 12, 13, 14, 15, 16, 17, 18, 19, 20, 21, 22, 23, 24, 25, 26, 27, 28 or 29 carbon atoms. The aliphatic hydrocarbon advantageously has 1 to 20 carbon atoms, more advantageously 1 to 18 carbon atoms, in particular 1 to 16 carbon atoms.

Aromatic hydrocarbons mean in particular benzene and its derivatives, for example toluene and xylene.

According to a further alternative or additional embodiment of the use claimed herein or of the method described herein, the organosilicon compound is a silicone.

In the context of the present invention, the term “silicone” refers to polymeric and oligomeric siloxanes. Siloxanes are saturated silicon-oxygen hydrides with unbranched or branched chains in which silicon and oxygen atoms alternate. Therefore, each silicon atom is separated from its nearest silicon neighbors by individual oxygen atoms. Unbranched siloxanes have the general structure H₃Si[OSiH₂]ₘOSiH₃. An example of a branched siloxane is H₃Si[OSiH₂]ₘOSiH[OSiH₂OSiH₃]₂. In the present case, hydrocarbyl derivatives are included. Examples of hydrocarbyl derivatives are linear siloxanes such as hexamethyldisiloxane, octamethyltrisiloxane, decamethyltetrasiloxane and polydimethylsiloxane, as well as cyclic siloxanes such as octamethylcyclotetrasiloxane, decamethylcyclopentasiloxane and dodecamethylcyclohexasiloxane.

According to another embodiment, the solvents contained in a solvent mixture S_{P} can be mixed with each other.

In the context of the present invention, two solvents are referred to as miscible if they are miscible at least during the respective reaction, that is, are not present as two phases.

In another embodiment of the use described herein or of the method claimed herein, R is selected from the group consisting of primary, secondary, tertiary alkyl, alkenyl and alkynyl radicals having 1 to 10 carbon atoms, cyclic alkyl radicals having 3 to 10 carbon atoms, a benzyl radical, mononuclear aryl radicals, polynuclear aryl radicals, mononuclear heteroaryl radicals and polynuclear heteroaryl radicals. The radical R may therefore also be primary, secondary, tertiary alkyl, alkenyl and alkynyl radicals having 2, 3, 4, 5, 6, 7, 8 or 9 carbon atoms, and cyclic alkyl radicals having 4, 5, 6, 7, 8 or 9 carbon atoms. According to another embodiment of the method, R is selected from the group consisting of primary, secondary, tertiary alkyl, alkenyl and alkynyl radicals having 1 to 6 carbon atoms, cyclic alkyl radicals having 3 to 6 carbon atoms, a benzyl radical, mononuclear aryl radicals, polynuclear aryl radicals, mononuclear heteroaryl radicals and polynuclear heteroaryl radicals. The radical R may therefore also be primary, secondary, tertiary alkyl, alkenyl and alkynyl radicals having 2, 3, 4 or 5 carbon atoms, and cyclic alkyl radicals having 4 or 5 carbon atoms. In yet another variant of the method, R is selected from the group consisting of Me, Et, n-Pr, i-Pr, n-Bu, i-Bu, s-Bu, t-Bu, n-pentyl, 2-pentyl, 3-pentyl, 2-methylbutyl, 3-methylbutyl, 3-methylbut-2-yl, 2-methylbut-2-yl, 2,2-dimethylpropyl, cyclopentyl, cyclohexyl, phenyl, tolyl, benzyl and cumyl, and isomers thereof. According to another advantageous embodiment, R is selected from the group consisting of Me, Et, n-Pr, i-Pr, n-Bu, i-Bu, s-Bu, t-Bu, cyclohexyl, phenyl, tolyl, benzyl and cumyl, and isomers thereof; R is particularly advantageously selected from the group consisting of Me, Et, n-Pr, i-Pr, n-Bu, i-Bu, s-Bu, t-Bu, in particular R=Me.

In a further embodiment of the use claimed herein or of the method described herein, it is provided that in step A.
an isomerically pure compound according to formula I or formula II
or
an isomer mixture,
comprising a first regioisomer according to formula I and a second regioisomer according to formula II, in particular consisting of a first regioisomer according to formula I and a second regioisomer according to formula II,
is provided.

The term “isomerically pure” has already been defined above. The isomerically pure compound is an alkali metal 8-R-dihydroguaiazulenide according to formula I or an alkali metal 6-R-dihydroguaiazulenide according to formula II.

The first regioisomer of the isomer mixture is an alkali metal 8-R-dihydroguaiazulenide according to formula I. The second regioisomer of the isomer mixture is an alkali metal 6-R-dihydroguaiazulenide according to formula II. In an alternative or additional embodiment of the use claimed herein or the method described herein, the isomer ratio of first regioisomer: second regioisomer is ≥80:20 and <90:10, advantageously between 81:19 and 89:11, in particular between 82:18 and 88:12, for example 83:17 or 84:16 or 85:15 or 86:14 or 87:13.

The following reaction equation applies in the case that an isomer mixture consisting of a first regioisomer according to formula I and a second regioisomer according to formula II is used in step A. of the method described herein. The product is then a diastereomer mixture of the Pt(IV) semi-sandwich complex [PtMe₃(GuaR)], which in the present case contains the maximum possible number of configurational isomers. Four diastereomeric enantiomer pairs are then present as shown in the illustration below. In this case, (GuaR)¹⁻=7-iso-propyl-1,4-dimethyl-8-R-dihydroazulenyl anion (Gua-8-R)¹⁻ (lower part of the illustration, left half) or 7-iso-propyl-1,4-dimethyl-6-R-dihydroazulenyl anion (Gua-6-Me)¹⁻ (lower part of the illustration, right half).

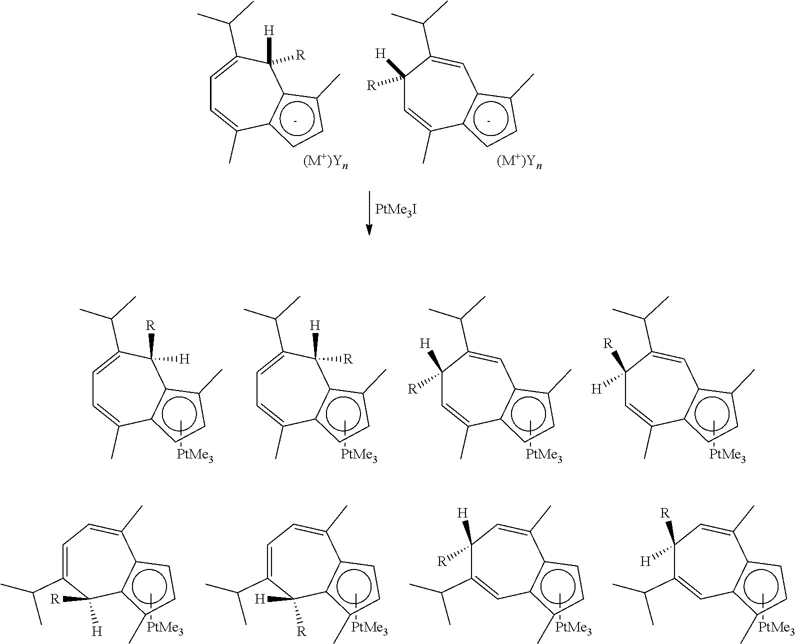

The following are shown from left to right in the upper row of the products: [PtMe₃(Gua-8-exo-R)] (III.D1), [PtMe₃(Gua-8-endo-R)] (III.D2), [PtMe₃(Gua-6-endo-R)] (IV.D3), [PtMe₃(Gua-6-exo-R)] (IV.D4). The respective associated enantiomer is shown in the lower row of the products, in each case directly below each of the aforementioned diastereomers.

Alternatively or in addition to trimethylplatinum(IV) iodide ([PtMe₃l]), which, as described in the literature, is present in the solid state as tetramer [PtMe₃I]₄, trimethylplatinum(IV) bromide ([PtMe₃Br]) and/or trimethylplatinum(IV) chloride ([PtMe₃Cl]) can be provided as platinum precursors. The latter are likewise present in the solid state as tetramers [PtMe₃Br]₄ or [PtMe₃Cl]₄.

According to a further embodiment of the use described herein or of the method claimed herein for producing platinum(IV) complexes according to general formula III and/or according to general formula IV, in step A the previously isolated and optionally purified compounds according to general formula I and/or general formula II are used.

The compound according to formula I and/or formula II can be provided in step A. as a substance, i.e., as a solid or liquid, or as a solution or suspension in a solvent, in particular a polar aprotic solvent. The latter is advantageously miscible with or identical to the solvent S_{P} provided in step B.

The term “miscible” has already been defined above.

Another embodiment of the use described herein or of the claimed method for producing platinum(IV) complexes according to formula III and/or formula IV provides that the provision in step A. comprises the in situ production of the compound according to general formula I and/or according to general formula II. In this case, the in situ production is carried out by reacting guaiazulene with an alkali metal organyl RM in a solvent S_{L}, which in particular comprises or is a polar aprotic solvent. R is as defined above, and M is an alkali metal, advantageously Li, Na or K.

In connection with the present invention, the phrase “produced in situ” or “in situ production” means that the reactants required for the synthesis of a compound to be produced in this way are reacted in a suitable stoichiometry in a solvent or mixture of solvents and the resulting product is not isolated. Instead, the solution or the suspension, which comprises the compound produced in situ, is generally reused directly, i.e., without isolation and/or further purification. The in situ production of a compound can take place in the reaction container provided for its further use or in a different reaction vessel. The terms “reaction vessel” and “reaction container” are already defined further above.

According to another embodiment of the use or method described herein, the provision in step A. comprises in situ production of the compound according to general formula I and/or according to general formula II, wherein a molar ratio of guaiazulene:alkali metal organyl RM is at least 1.00:2.00. It can therefore also be 1.00:1.00. In an alternative or additional embodiment, the molar ratio of guaiazulene:alkali metal organyl RM is between 1.00:2.00 and 2.00:1.00, advantageously between 1.00:1.75 and 1.75:1.00, in particular between 1.00:1.50 and 1.50:1.00, for example 1.00:1.95 or 1.95:1.00 or 1.00:1.90 or 1.90:1.00 or 1.00:1.85 or 1.85:1.00 or 1.00:1.80 or 1.80:1.00 or 1.00:1.70 or 1.70:1.00 or 1.00:1.65 or 1.65:1.00 or 1.00:1.60 or 1.60:1.00 or 1.00:1.55 or 1.55:1.00 or 1.00:1.45 or 1.45:1.00 or 1.00:1.40 or 1.40:1.00 or 1.00:1.35 or 1.35:1.00 or 1.00:1.30 or 1.30:1.00 or 1.00:1.25 or 1.25:1.00 or 1.00:1.20 or 1.20:1.00 or 1.00:1.15 or 1.15:1.00 or 1.00:1.10 or 1.10:1.00 or 1.00:1.05 or 1.05:1.00. In an advantageous embodiment, the molar ratio of guaiazulene:alkali metal organyl RM is 1.00:1.00.

A further variant of the claimed use or the claimed method provides that the in situ preparation of the compound according to general formula I and/or according to general formula II is carried out in step A. in a solvent or solvent mixture S_{L}, which is in particular miscible with or identical to the solvent S_{P} from step B. A definition of the term “miscible” has already been given above. Then, assuming the chemical inertness of the solvent S_{L} and the solvent S_{P} provided in step B, a solvent change can be dispensed with, which is particularly advantageous from a (method) economic and environmental point of view.

In a further variant, the solvents S_{P} and S_{L} are chemically inert.

In the context of the present invention, the term “chemically inert solvent” means a solvent that is not chemically reactive under the respective process conditions. Under the respective reaction conditions, including the purification and/or isolation steps, the inert solvent therefore does not react with a potential reaction partner, in particular not with a reactant and/or an intermediate and/or a product and/or a by-product, and not with another solvent, air, or water.

For example, the polar aprotic solvent S_{L} is an ether or comprises at least one ether. The ether can be selected from the group consisting of tetrahydrofuran, methyl tetrahydrofuran, 1,4-dioxane, 1,2-dimethoxyethane, diethyl ether, methyl tert-butyl ether, di-n-propyl ether, di-iso-propyl ether, cyclopentyl methyl ether, and isomers thereof, and mixtures thereof.

According to an alternative or additional embodiment of the use claimed herein or of the method described herein, the solvent S_{L} comprises or is at least one organosilicon compound, in particular a silicone. The term “silicone” has already been defined above. In addition, examples of silicones are provided in a non-limiting manner.

In another variant of the method for producing platinum(IV) complexes according to formulae III and IV, it is provided that the synthesis in step B. comprises at least one salt metathesis reaction.

Yet another embodiment of the method claimed herein provides that the platinum precursor to be provided in step A. is selected from the group consisting of trimethylplatinum(IV) iodide, trimethylplatinum(IV) bromide and trimethylplatinum(IV) chloride, and mixtures thereof, wherein provision takes place
i. as a solution comprising the platinum precursor and a solvent SA that is in particular miscible with or identical to the solvent S_{P}
or
ii. as a solid.

The solvent SA is advantageously a polar aprotic solvent or a solvent mixture. It is particularly advantageous if the solvent SA comprises or is an ether. The ether is selected, for example, from the group consisting of tetrahydrofuran, methyl tetrahydrofuran, 1,4-dioxane, 1,2-dimethoxyethane, diethyl ether, methyl tert-butyl ether, di-n-propyl ether, di-iso-propyl ether, cyclopentyl methyl ether, and isomers thereof, and mixtures thereof.

Another embodiment of the use or of the method provides that the alkali metal cation M⁺ is selected from the group consisting of Li⁺, Na⁺ and K⁺.

According to a further variant of the method claimed herein, it is provided that the neutral ligand Y is a polar aprotic solvent. The polar aprotic solvent is advantageously selected from the group consisting of alkoxyalkanes, thioethers and tertiary amines, in particular from the group consisting of alkoxyalkanes and thioethers. The terms “alkoxyalkane” and “thioether” are already defined further above.

In another variant of the method claimed herein, the neutral ligand Y is a crown ether selected from the group consisting of macrocyclic polyethers and aza-, phospha- and thia-derivatives thereof, wherein an inner diameter of the crown ether and an ion radius of the respective alkali metal cation M⁺ correspond to each other. A definition of the term “crown ether” has already been given above.

According to a further embodiment of the method claimed herein, a glycol dialkyl ether is provided as a neutral ligand Y, selected from the group consisting of a monoethylene glycol dialkyl ether, a diethylene glycol dialkyl ether, a triethylene glycol dialkyl ether, a monopropylene glycol dialkyl ether, a dipropylene glycol dialkyl ether, a tripropylene glycol dialkyl ether, a monooxomethylene dialkyl ether, a dioxomethylene dialkyl ether and a trioxomethylene dialkyl ether, the isomer mixtures thereof, and mixtures thereof. A selection of usable glycol dialkyl ether is indicated further above in a non-limiting manner.

A further embodiment of the method claimed herein provides that the neutral ligand Y is an ether. For example, the ether can be a non-cyclic or a cyclic ether selected from the group consisting of dialkyl ethers, cyclopentyl methyl ether, tetrahydrofuran, 2-methyltetrahydrofuran, 3-methyltetrahydrofuran, tetrahydropyran, 1,4-dioxane, 1,2-dimethoxyethane, and isomers thereof, and mixtures thereof, in particular from the group consisting of diethyl ether, methyl tert-butyl ether, di-n-propyl ether, di-iso-propyl ether, di-n-butyl ether, di-iso-butyl ether, di-tert-butyl ether, tetrahydrofuran, 2-methyltetrahydrofuran, 3-methyltetrahydrofuran, tetrahydropyran, 1,4-dioxane, 1,2-dimethoxyethane, and isomers thereof, and mixtures thereof.

It is particularly advantageous that the use of compounds according to general formula I and/or general formula II for producing platinum(IV) complexes according to general formula III and/or general formula IV or the method for producing such platinum(IV) complexes using compounds according to general formula I and/or general formula II allows the preparation of high-purity platinum(IV) complexes. In particular, by means of the method described herein, a plurality of diastereomer mixtures, in particular of platinum(IV) complexes, that are liquid at room temperature, can be provided in a good yield of typically 60%, advantageously 70%, and in a good space-time yield. One example of such a diastereomer mixture is [PtMe₃(GuaMe)]. In general, the end product may still contain residues of solvents or for example impurities from the reactants. It is known to a person skilled in the art that the content of impurities, for example solvents, can be determined by gas chromatography (GC) methods, optionally with mass spectrometry coupling (GC-MS).

Definitions of the terms “high-purity platinum(IV) complex” or “platinum(IV) complex in high purity” and the term “space-time yield” are given further above.

By means of the method described herein, the platinum(IV) semi-sandwich complex [PtMe₃(GuaMe)], for example, can be produced, for example starting from the platinum precursor [PtMe₃I]₄ and an isomer mixture of a lithium dihydroguaiazulenide, consisting of a first regioisomer according to formula I.1 and a second regioisomer according to formula II.1:

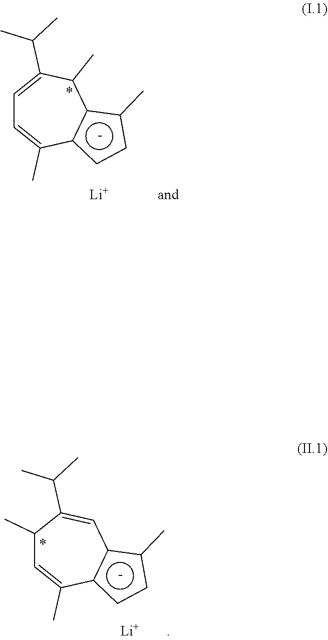

The reaction equation for the preparation of [PtMe₃(GuaMe)] is then:

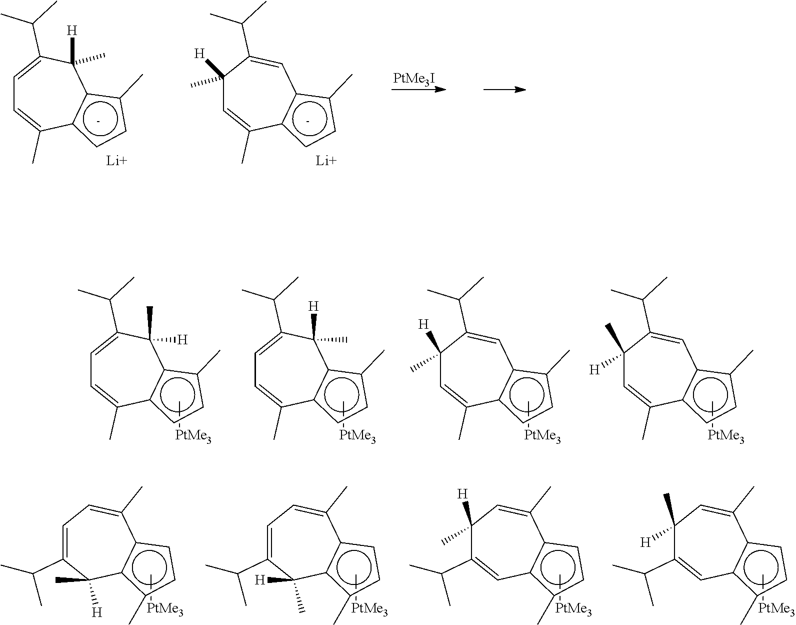

In this reaction regime, the platinum(IV) complex [PtMe₃(GuaMe)] is obtained as diastereomer mixture consisting of eight configurational isomers resulting from the presence of planar and central chirality, namely four diastereomers and enantiomers thereof, specifically in the form of an oil. The yield is usually >75%, for example 77%.

If a purification of the oil obtained is desired or necessary, this can advantageously be carried out by simple sublimation and/or distillation. Further purification of the product, which is not absolutely necessary depending on the choice of the reactants, reaction conditions and solvents for producing the platinum(IV) complex and/or the use of the platinum(IV) complex, can be carried out by means of column chromatography, for example via silica or Al₂O₃ (neutral) with a non-polar aprotic solvent, for example hexane, as an eluent. In the absence of light, the monomeric compound [PtMe₃(GuaMe)] can be stored at room temperature for several months. In the meantime, no decomposition reactions, oligomerization or polymerization are observed.

Only thin-layer chromatography provides indications of the four possible diastereomers [PtMe₃(Gua-6-endo-Me)], [PtMe₃(Gua-6-exo-Me)], [PtMe₃(Gua-8-endo-Me)] and [PtMe₃(Gua-8-exo-Me)], which are each present as enantiomer mixtures. By means of ¹H-NMR spectroscopy, only signals of the diastereomers are detected, which are obtained starting from the lithium dihydroguaiazulenide according to formula I.1, i.e., the diastereomers according to the formula [PtMe₃(Gua-8-Me)] or according to the formulae [PtMe₃(Gua-8-exo-Me)] (III.D1.1) and [PtMe₃(Gua-8-endo-Me)] (III.D2.1). The latter are shown below:

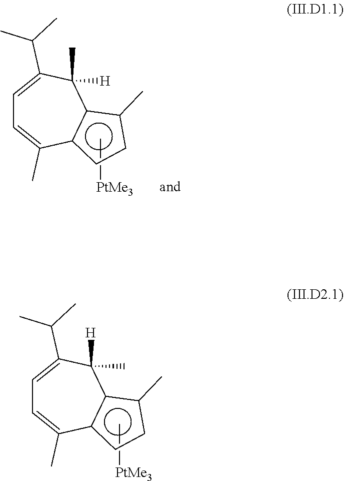

The diastereomer according to formula III.D1.1 is the main diastereomer. For example, ¹H-NMR spectroscopy was used to determine a diastereomeric ratio d.r. for first diastereomer (III.D1.1): second diastereomer of 68:32 (III.D2.1). The diastereomers obtained starting from the lithium dihydroguaiazulenide according to formula II.1 according to the formula [PtMe₃(Gua-6-Me)] or according to the formulae PtMe₃(Gua-6-endo-Me) (IV.D3.1) and [PtMe₃(Gua-6-exo-Me)] (IV.D4.1) were detected only by means of thin-layer chromatography. They are shown below:

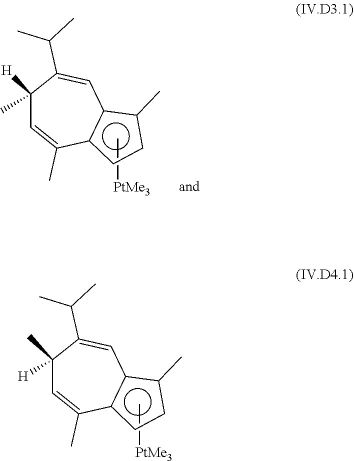

The fact that the platinum(IV) complex obtainable by means of the method described herein is present as a liquid in an isolated form is advantageous in particular with regard to use as a platinum precursor compound for vapor deposition processes, in particular low-temperature vapor deposition processes. It is also noteworthy that the resulting complex [PtMe₃(GuaMe)] has a relatively high thermal stability. According to thermogravimetric analysis (TGA), 3% degradation occurs at a temperature of 180° C.

In addition, this platinum(IV) compound, which is liquid at room temperature, can be advantageously used as a precatalyst and/or catalyst in catalysis, for example for light-induced platinum-catalyzed hydrosilylation reactions, the hydrogenation of unsaturated compounds, and polymerization reactions in which the activation is effected by ultraviolet or visible radiation. This is because the platinum(IV) complex [PtMe₃(GuaMe)] is easier to handle than previously known platinum(IV) compounds which contain cyclopentadienyl anions and are frequently present in a waxy form, such as [PtMe₃(MeCp)] due to the fact that it is obtained in the form of an oil. The platinum(IV) compound [PtMe₃(GuaMe)] also shows absorption in the VIS range. This is a further advantage in the context of light-induced platinum-catalyzed reactions, for example hydrosilylation reactions. This is because the use of UV/VIS light is regularly provided, as a result of which special safety measures are usually required in order to reduce the risk of skin cancer. Such safety measures are not absolutely necessary when [PtMe₃(GuaMe)] is used.

The solution present after the synthesis in step B., which comprises the target compound according to general formula III and/or formula IV in solution, can be directly reacted with one or more further reactants. Alternatively, after the reaction, a step C. is carried out, which comprises isolating the platinum(IV) complex according to formula III and/or formula IV:
as a solution comprising the platinum(IV) complex according to formula III and/or according to formula IV and the solvent S_{P},
or
as a solid or liquid.

The isolation of the platinum(IV) complex according to formula III and/or formula IV as a solution, as a solid or as a liquid may comprise one or more method steps, such as one or more filtration steps, the reduction of the mother liquor volume, i.e., concentration, for example by means of “bulb-to-bulb,” the addition of a solvent and/or a solvent exchange to precipitate the product from the mother liquor and/or to remove impurities and/or reactants, sublimation, distillation, a column chromatographic purification, washing and drying of the product. Optionally, filtration over a cleaning medium, for example activated carbon or silica, e.g. Celite®, can be carried out. The aforementioned steps may each be provided in different orders and frequencies.

The object is also achieved by
a platinum(IV) complex according to general formula

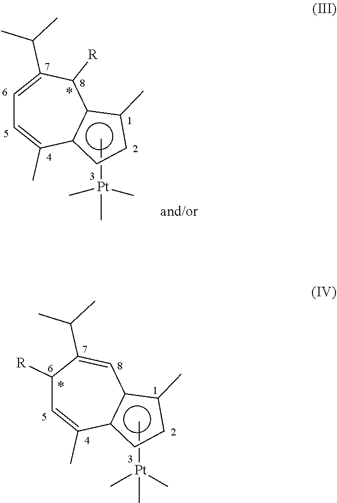

or
a solution or a suspension comprising a platinum(IV) complex according to general formula

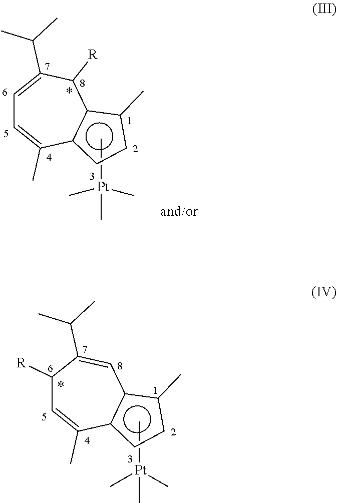

and a solvent that is in particular miscible with or identical to the solvent S_{P}, wherein
R is in each case selected from the group consisting of primary, secondary, tertiary alkyl, alkenyl and alkynyl radicals having 1 to 10 carbon atoms, cyclic alkyl radicals having 3 to 10 carbon atoms, a benzyl radical, mononuclear aryl radicals, polynuclear aryl radicals, mononuclear heteroaryl radicals and polynuclear heteroaryl radicals.

Moreover, the object is achieved by
a platinum(IV) complex according to general formula

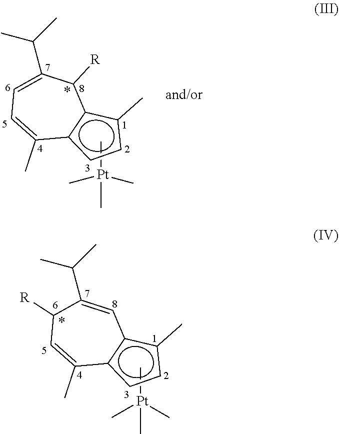

or
a solution or a suspension comprising a platinum(IV) complex according to general formula

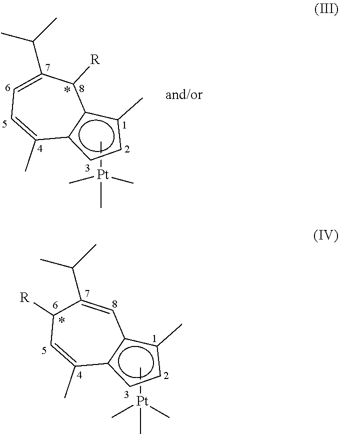

and a solvent that is in particular miscible with or identical to the solvent S_{P},
wherein
R is in each case selected from the group consisting of primary, secondary, tertiary alkyl, alkenyl and alkynyl radicals having 1 to 10 carbon atoms, cyclic alkyl radicals having 3 to 10 carbon atoms, a benzyl radical, mononuclear aryl radicals, polynuclear aryl radicals, mononuclear heteroaryl radicals and polynuclear heteroaryl radicals,
obtained or obtainable according to a method for producing such a platinum(IV) complex according to one of the embodiments described above.

In this case, the general formulae III and IV each comprise both the monomers and any oligomers, in particular dimers, and solvent adducts. In addition, the claimed compounds according to general formulae III and IV are in particular each present as a diastereomer mixture. In other words: In the case of the platinum(IV) complex according to formula III, the carbon atom C8 is a stereocenter. In the case of the platinum(IV) complex according to formula IV, the carbon atom C6 is a stereocenter.

Alternatively, a diastereomer mixture comprising, in particular consisting of, the diastereomer mixture of the compound according to formula III and the diastereomer mixture of the compound according to formula IV, can be obtained, in particular as a function of the selected substituent R.

In each of the aforementioned cases, each diastereomer is present as an enantiomer mixture, wherein the diastereomers can be present independently of one another as a racemate.

The platinum(IV) complexes according to general formulae III and IV are usually obtained solvent-free, i.e., not as solvent adducts, or are generally present in a solvent-free form. However, by means of the method described further above for producing such platinum(IV) complexes, solvent adducts of these metal complexes can also be obtained. In the case of such an adduct, the solvent is in particular identical to the solvent S_{P} used in the context of the method described further above, in particular if the solvent S_{P} is an alkoxyalkane or the solvent S_{P} comprises an alkoxyalkane.

The platinum(IV) complex according to the general formulae III and IV claimed herein each have an organo-dihydroazulenyl anion or R-dihydroguaiazulenyl anion (GuaR)¹⁻, which carries an organyl radical R in the 8 position or in the 6 position of the guaiazulene skeleton in addition to an H atom. The R-dihydroguaiazulenyl anion (GuaR)¹⁻ can therefore be a 7-iso-propyl-1,4-dimethyl-8-R-dihydroazulenyl anion or 8-R-dihydroguaiazulenyl anion (Gua-8-R)¹⁻ according to formula III or a 7-iso-propyl-1,4-dimethyl-6-R-dihydroazulenyl anion or 6-R-dihydroguaiazulenyl anion (Gua-6-R)¹⁻ according to formula IV. In other words: There is an RCH group in the C8 position or in the C6 position of the guaiazulene skeleton. As a result of the addition of an organyl anion (R)¹⁻, the aromaticity is limited to the five-membered ring, wherein the blue color typical for azulene and derivatives thereof is generally lost. The organo-dihydroazulenyl ligand anion (GuaR)¹⁻ is a derivative of the cyclopentadienyl anion or a cyclopentadienyl-like monoanion.

Owing to the presence of an R-dihydroguaiazulenyl anion, i.e., of a cyclopentadienyl-like monoanion, the compounds according to general formulae III and IV are suitable in particular as precatalysts and/or as catalysts for chemical reactions in which platinum(IV) complexes having cyclopentadienyl ligands are otherwise used. This is particularly advantageous because the provision of an R-dihydroguaiazulenyl ligand is less labor-intensive and time-consuming compared to the provision of the cyclopentadienyl ligand. In particular, the compounds used as reactants herein, which comprise cyclopentadienyl-like ligands, can be prepared using inexpensive renewable raw materials. This is because guaiazulene is partially accessible synthetically, namely starting from the natural material guaiacol and other azulene formers by simple dehydration and dehydrogenation (T. Shono, N. Kise, T. Fujimoto, N. Tominaga, H. Morita, J. Org. Chem. 1992, 57, 26, 7175-7187; CH 314 487 A (B. Joos) Jan. 29, 1953). As a result, the synthesis effort and the production costs for the platinum(IV) complexes claimed herein, as well as for solutions or suspensions comprising such a compound and a solvent, which is in particular miscible with or identical to the solvent S_{P}, are also lower than for analogous platinum(IV)-Cp complexes. Consequently, the platinum(IV) complexes described herein and their solutions and suspensions represent a relatively cost-effective and, in particular, sustainable alternative to platinum(IV) cyclopentadienyl complexes, particularly with respect to an industrial application.

Guaiazulene is a natural substance that contains chamomile oil and other essential oils and is therefore advantageously inexpensive to obtain in large quantities. It can be produced synthetically from the guaiacol of guaiac wood oil (guaiac resin). Guaiazulene is an intensely blue substance with anti-inflammatory properties.

In one embodiment of the compounds according to formula III and/or formula IV claimed herein, R is selected from the group consisting of primary, secondary, tertiary alkyl, alkenyl and alkynyl radicals having 1 to 10 carbon atoms, cyclic alkyl radicals having 3 to 10 carbon atoms, a benzyl radical, mononuclear aryl radicals, polynuclear aryl radicals, mononuclear heteroaryl radicals and polynuclear heteroaryl radicals. The radical R may therefore also be primary, secondary, tertiary alkyl, alkenyl and alkynyl radicals having 2, 3, 4, 5, 6, 7, 8 or 9 carbon atoms, and cyclic alkyl radicals having 4, 5, 6, 7, 8 or 9 carbon atoms. According to another embodiment of the compounds according to formula III and/or formula IV claimed herein, R is selected from the group consisting of primary, secondary, tertiary alkyl, alkenyl and alkynyl radicals having 1 to 6 carbon atoms, cyclic alkyl radicals having 3 to 6 carbon atoms, a benzyl radical, mononuclear aryl radicals, polynuclear aryl radicals, mononuclear heteroaryl radicals and polynuclear heteroaryl radicals. The radical R may therefore also be primary, secondary, tertiary alkyl, alkenyl and alkynyl radicals having 2, 3, 4 or 5 carbon atoms, and cyclic alkyl radicals having 4 or 5 carbon atoms. In yet another variant of the claimed compounds, R is selected from the group consisting of Me, Et, n-Pr, i-Pr, n-Bu, i-Bu, s-Bu, t-Bu, n-pentyl, 2-pentyl, 3-pentyl, 2-methylbutyl, 3-methylbutyl, 3-methylbut-2-yl, 2-methylbut-2-yl, 2,2-dimethylpropyl, cyclopentyl, cyclohexyl, phenyl, tolyl, benzyl and cumyl, and isomers thereof. According to a further advantageous embodiment, R is selected from the group consisting of Me, Et, n-Pr, i-Pr, n-Bu, i-Bu, s-Bu, t-Bu, cyclohexyl, phenyl, tolyl, benzyl and cumyl, and isomers thereof; R is particularly advantageously selected from the group consisting of Me, Et, n-Pr, i-Pr, n-Bu, i-Bu, s-Bu, t-Bu, in particular R=Me.

According to a further embodiment of the claimed platinum(IV) complexes or solutions or suspensions, a diastereomer mixture is present, comprising
a first diastereomer according to formula III.D1 and a second diastereomer according to formula III.D2,

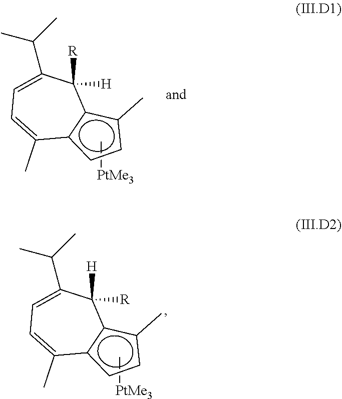

and/or
a third diastereomer according to formula IV.D3 and a fourth diastereomer according to formula IV.D4,

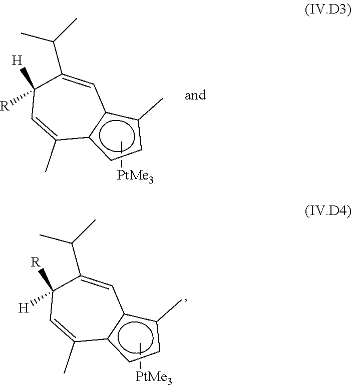

wherein each diastereomer is present as an enantiomer mixture.

In this case, the first and the second diastereomers each have an RCH group in the C8 position of the guaiazulene skeleton, and the third and the fourth diastereomers have an RCH group in each case in the C6 position of the guaiazulene skeleton. In other words: In the case of the first diastereomer (III.D1) and the second diastereomer (III.D2), the carbon atom C8 represents a stereocenter in each case. In the case of the third diastereomer (IV.D3) and the fourth diastereomer (IV.D4), the carbon atom C6 represents a stereocenter in each case. In addition, each of the four possible diastereomers is present as an enantiomer mixture. Consequently, a diastereomer mixture of a platinum(IV) complex claimed herein has at least four stereoisomers, namely two diastereomers and the total two enantiomers thereof. In other words: A platinum(IV) complex described herein can be present as a mixture of four or eight configurational isomers, i.e., as a mixture of two or four diastereomers plus one enantiomer per diastereomer.

In another embodiment of the platinum(IV) complexes or solutions or suspensions claimed herein, at least one diastereomer is present as a racemate. The diastereomers can thus be present independently of one another as racemates.

According to yet another embodiment of the platinum(IV) complexes or solutions or suspensions described herein, a mixture of two diastereomers is present, wherein a diastereomer ratio of
first diastereomer: second diastereomer
or
third diastereomer: fourth diastereomer
is ≥60:40 and <90:10, advantageously between 61:39 and 89:11, more advantageously between 62:38 and 88:12, in particular between 63:37 and 87:13, for example 64:36 or 65:35 or 66:34 or 67:33 or 68:32 or 69:31 or 70:30 or 71:29 or 72:28 or 73:27 or 74:26 or 75:25 or 76:24 or 77:23 or 78:22 or 79:21 or 80:20 or 81:19 or 82:18 or 83:17 or 84:16 or 85:15 or 86:14 or 87:13.

The respective diastereomer ratio d.r. is determined, for example, by means of nuclear magnetic resonance spectroscopy, in particular by means of ¹H-NMR spectroscopy.

According to a further advantageous embodiment of the platinum(IV) complexes or solutions or suspensions claimed herein, a diastereomer mixture is present, consisting of the first diastereomer according to formula III.D1 and the second diastereomer according to formula III.D2,

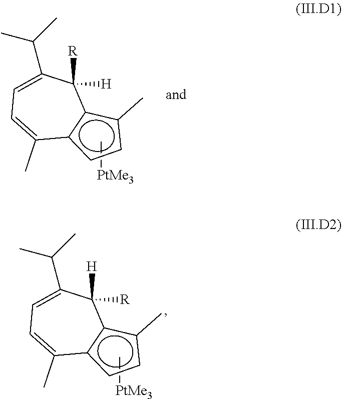

wherein each diastereomer is present as an enantiomer mixture.

The aforementioned diastereomer mixture is the diastereomer mixture of the platinum(IV) complex according to formula III and the diastereomer mixture of the compound [PtMe₃(Gua-8-R)]. This diastereomer mixture consists of exactly four configuration isomers according to the following illustration, namely of the two diastereomers [PtMe₃(Gua-8-exo-R)] and [PtMe₃(Gua-8-endo-R)] (upper row, from left to right) and their enantiomers (lower row, from left to right).

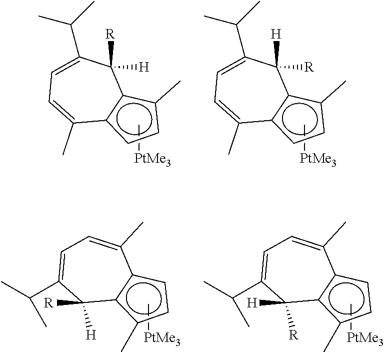

According to a further advantageous embodiment of the platinum(IV) complexes claimed herein or solutions or suspensions, R=Me. In this case, the platinum(IV) complex is present as a mixture of two diastereomers, namely a first diastereomer according to formula III.D1.1 and a second diastereomer according to formula III.D2.1, in particular in an isolated form as a diastereomer mixture that is liquid at room temperature. The illustration below shows the two aforementioned diastereomers:

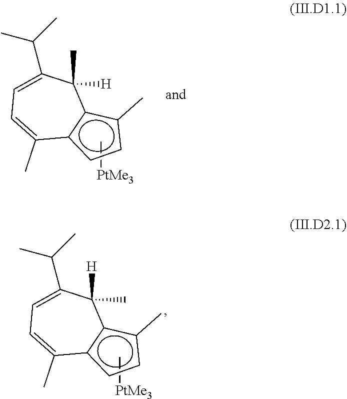

According to an alternative or additional embodiment, the diastereomer ratio of first diastereomer (formula III.D1.1): second diastereomer (formula III.D2.1) is between 65:35 and 75:25, for example 68:32, wherein each diastereomer is present as an enantiomer mixture, in each case optionally as a racemate independently of one another.

According to another embodiment of the platinum(IV) complexes or solutions or suspensions claimed herein, R=Me and the platinum(IV) complex [PtMe₃(GuaMe)] is present as a mixture of four diastereomers, in particular in an isolated form as a diastereomer mixture that is liquid at room temperature, consisting of four diastereomeric enantiomer pairs, as shown below. In this case, (GuaMe)¹⁻=7-iso-propyl-1,4,8-trimethyl-dihydroazulenyl anion (Gua-8-Me)¹⁻ (lower half of the illustration) or 7-iso-propyl-1,4,6-trimethyl-dihydroazulenyl anion (Gua-6-Me)¹⁻ (right half of the illustration.).

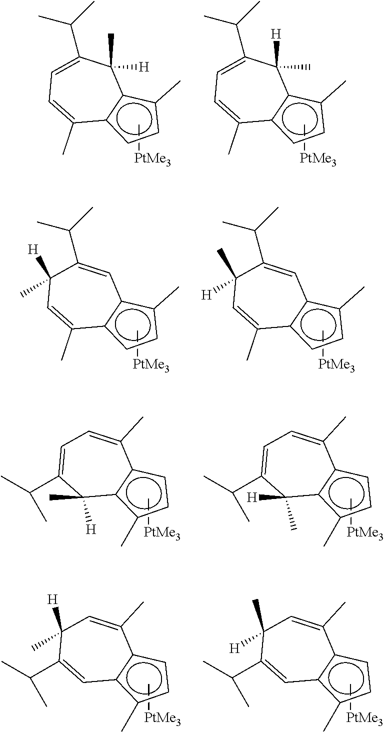

In the upper row from left to right, the illustration shows: [PtMe₃(Gua-8-exo-Me)] (III. D1.1), [PtMe₃(Gua-8-endo-Me)] (III.D2.1), [PtMe₃(Gua-6-endo-Me)] (IV.D3.1), [PtMe₃(Gua-6-exo-Me)] (IV.D4.1). The respective associated enantiomer is shown in the lower row, in each case directly below each of the aforementioned diastereomers. According to an alternative or additional embodiment, the diastereomeric ratio of first diastereomer (formula III.D1.1): second diastereomer (formula III.D2.1) is between 65:35 and 75:25, for example 68:32, wherein each diastereomer is optionally present as a racemate.

The fact that the platinum(IV) complex [PtMe₃(GuaMe)] described herein is present in an isolated form as a diastereomer mixture that is liquid at room temperature is particularly advantageous with regard to use as a platinum precursor compound for vapor deposition processes, in particular low-temperature vapor deposition processes. It is also noteworthy that the complex [PtMe₃(GuaMe)] has a relatively high thermal stability. According to thermogravimetric analysis (TGA), 3% degradation occurs at a temperature of 180° C.

In addition, this platinum(IV) compound, which is liquid at room temperature, can be advantageously used as a precatalyst and/or catalyst in catalysis, for example for light-induced platinum-catalyzed hydrosilylation reactions, the hydrogenation of unsaturated compounds, and polymerization reactions in which the activation is effected by ultraviolet or visible radiation. This is because the platinum(IV) complex [PtMe₃(GuaMe)] is easier to handle than previously known platinum(IV) compounds containing cyclopentadienyl anions and frequently present in a waxy form, such as [PtMe₃(MeCp)] due to the fact that it is obtained or present in the form of an oil. The platinum(IV) compound [PtMe₃(GuaMe)] also shows absorption in the VIS range. This is a further advantage in the context of light-induced platinum-catalyzed reactions, for example hydrosilylation reactions. This is because the use of UV/VIS light is regularly provided, as a result of which special safety measures are usually required in order to reduce the risk of skin cancer. Such safety measures are not absolutely necessary when [PtMe₃(GuaMe)] is used. Furthermore, it is advantageous that the monomeric compound [PtMe₃(GuaMe)] can be stored at room temperature for several months in the absence of light. In the meantime, no decomposition reactions, oligomerization or polymerization are observed.

According to an advantageous embodiment, the isolated platinum(IV) complex according to formula III and/or the formula IV has a total content of impurities, comprising in particular impurities due to reactants, by-products, atmospheric oxygen, water, oxygen-containing compounds, semi-metals, metals, in particular platinum(0), optionally in the form of platinum(0) nanoparticles and/or nanoparticles containing platinum(0), and solvents, of below 1000 ppm, ideally of below 100 ppm.

The platinum(IV) complexes according to formula III and/or IV and the solutions or suspensions thereof in a solvent, which is in particular miscible with or identical to the solvent S_{P}, for example S_{L}, can be obtained, advantageously in a simple, reproducible and comparatively cost-effective manner, in a high purity of 97%, advantageously higher than 97%, in particular higher than 98% or 99%, and in a good yield of typically ≥60%, advantageously ≥70%, and in a good space-time yield, in particular by means of the method described further above for producing such platinum(IV) complexes, solutions and suspensions. In general, the end product may still contain residues of solvents or for example impurities from the reactants. It is known to a person skilled in the art that the content of impurities, for example solvents, can be determined by gas chromatography (GC) methods, optionally with mass spectrometry coupling (GC-MS).

Definitions of the terms “platinum(IV) complex in a high purity” and “space-time yield” are already given above.

Finally, the platinum(IV) complexes according to general formulae III and IV and solutions or suspensions claimed herein comprising at least one such platinum(IV) complex are suitable as high-quality reactants for further reactions and/or applications, even on an industrial scale.

Moreover, the object is achieved by
a platinum(IV) complex according to formula

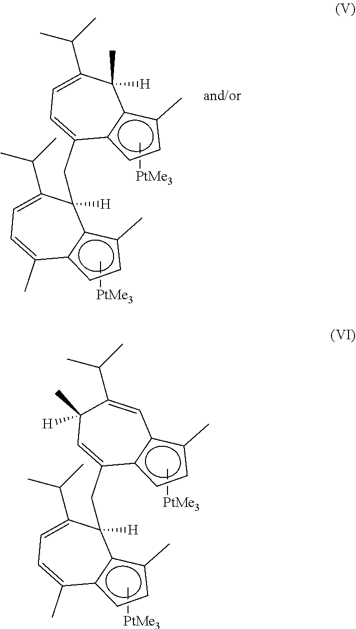

or
a solution or a suspension comprising a platinum(IV) complex according to formula V and/or formula VI and a solvent SD that comprises or is a nonpolar aprotic or a polar aprotic solvent.

The platinum(IV) complexes according to formula V and formula VI are each the product of a photoinduced, in particular daylight-induced, dimerization. The compounds according to formula V and formula VI each have two 4,8-linked organo-dihydroguaiazulenyl radical anions. In this case, the radical anion involved in the linkage via its C4 atom in the 8 position or in the 6 position of the guaiazulene skeleton carries, in addition to an H atom, an organyl radical R, in particular a methyl radical. Formulae V and VI each also comprise solvent adducts.

According to one embodiment of the platinum(IV) complexes according to formula V and formula VI or solutions or suspensions claimed herein comprising at least one such platinum(IV) complex, the solvent SD comprises at least one solvent selected from the group consisting of polar aprotic solvents, aliphatic hydrocarbons, aromatic hydrocarbons, organosilicon compounds, and mixtures thereof. The solvent SD is advantageously selected from the group consisting of polar aprotic solvents, aliphatic hydrocarbons, in particular having 1 to 30 carbon atoms, aromatic hydrocarbons, organosilicon compounds, and mixtures thereof.

The polar aprotic solvent is, for example, an ether or comprises at least one ether. The ether can be selected from the group consisting of tetrahydrofuran, methyl tetrahydrofuran, 1,4-dioxane, 1,2-dimethoxyethane, diethyl ether, methyl tert-butyl ether, di-n-propyl ether, di-iso-propyl ether, cyclopentyl methyl ether, and isomers thereof, and mixtures thereof.

A definition of the term “aliphatic hydrocarbons” has already been given above. The aliphatic hydrocarbon can also have 2, 3, 4, 5, 6, 7, 8, 9, 10, 11, 12, 13, 14, 15, 16, 17, 18, 19, 20, 21, 22, 23, 24, 25, 26, 27, 28 or 29 carbon atoms. The aliphatic hydrocarbon advantageously has 1 to 20 carbon atoms, more advantageously 1 to 18 carbon atoms, in particular 1 to 16 carbon atoms.

Aromatic hydrocarbons mean in particular benzene and its derivatives, for example toluene and xylene.

According to a further alternative or additional embodiment of the platinum(IV) complexes or solutions or suspensions claimed herein comprising at least one such platinum(IV) complex, the organosilicon compound is a silicone. A definition of the term “silicone” has already been given above. In addition, examples of silicones are listed there in a non-limiting manner.

An example of a polar aprotic solvent is dimethyl sulfoxide (DMSO).

According to another embodiment, the solvents contained in a solvent mixture SD can be mixed with each other. The term “miscible” has already been defined above.

A further advantageous embodiment provides that the solvent SD is miscible with or identical to the solvent S_{P} defined further above.

The dimerization reaction takes place in solution, in particular in one of the abovementioned nonpolar aprotic solvents SD or in a polar aprotic solvent SD such as DMSO, as a result of a light-induced elimination of methane according to the following gross equation:

2[PtMe₃(GuaMe)]-hv->[Me₃Pt(Gua-Gua)PtMe₃]+CH₄←

The homodimer according to formula V can be obtained starting from a solution of a diastereomer mixture consisting of the two diastereomers PtMe₃(Gua-8-exo-Me)] (III.D1.1) and [PtMe₃(Gua-8-endo-Me)] (III.D2.1). The platinum(IV) complex according to formula V was identified by means of mass spectrometric analysis. The heterodimer according to formula VI can be obtained starting from a solution of a diastereomer mixture consisting of the four diastereomers [PtMe₃(Gua-8-exo-Me)] (III.D1.1), [PtMe₃(Gua-8-endo-Me)](III.D2.1), [PtMe₃(Gua-6-endo-Me)] (IV.D3.1), [PtMe₃(Gua-6-exo-Me)](IV.D4.1). The platinum(IV) complex according to formula VI was identified by means of X-ray structure analysis and mass spectrometry.

In both cases, the solvent is one of the abovementioned nonpolar aprotic solvents SD or a polar aprotic solvent SD, for example DMSO. Depending on the reaction conditions, in particular the concentration of the respective diastereomer mixture in the selected solvent and the intensity of the visible light used for irradiation, the reaction time ranges from 1 hour to 72 hours.

The object is also achieved by the use of
at least one platinum(IV) complex according to general formula

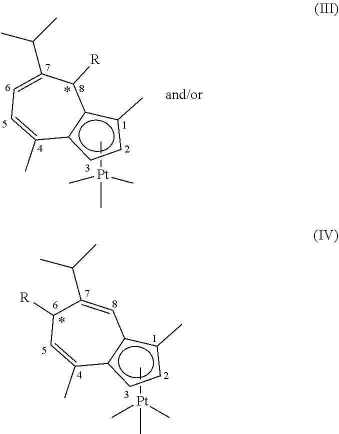

and/or
according to formula

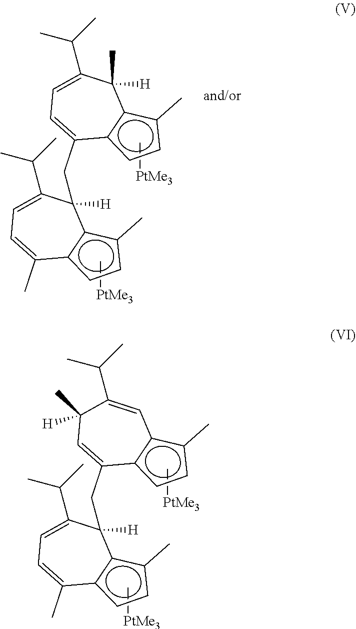

or
at least one solution or suspension comprising a platinum(IV) complex according to general formula

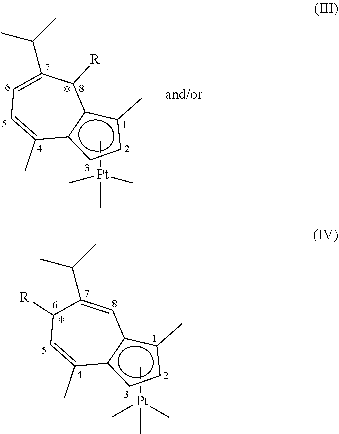

and a solvent that is in particular miscible with or identical to the solvent S_{P}
and/or
comprising a platinum(IV) complex according to formula

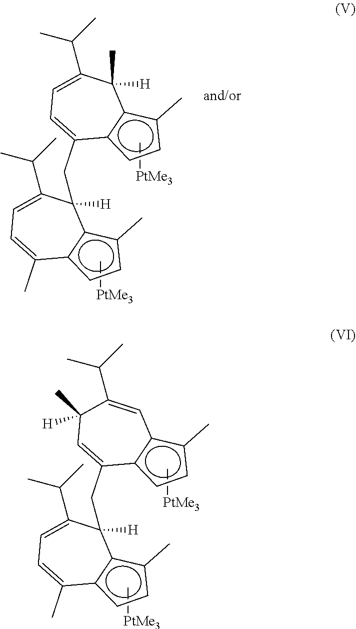

and a solvent SD that comprises or is a nonpolar aprotic or a polar aprotic solvent, in particular is miscible with or identical to the solvent S_{P},
wherein
R is in each case selected from the group consisting of primary, secondary, tertiary alkyl, alkenyl and alkynyl radicals having 1 to 10 carbon atoms, cyclic alkyl radicals having 3 to 10 carbon atoms, a benzyl radical, mononuclear aryl radicals, polynuclear aryl radicals, mononuclear heteroaryl radicals and polynuclear heteroaryl radicals,
   according to an embodiment of the platinum(IV) complexes described above
or
   according to an embodiment of the solutions or suspensions described above, comprising such a platinum(IV) complex,
or
   obtained or obtainable according to a method for producing such platinum(IV) complexes or solutions or suspensions according to any one of the embodiments described further above,
as
i. a precatalyst or a solution or suspension containing a precatalyst in a chemical reaction
and/or
ii. a catalyst or a solution or suspension containing a catalyst in a chemical reaction
and/or
iii. a precursor compound or a solution or suspension containing a precursor compound for producing at least one layer consisting of platinum or at least one layer containing platinum on at least one surface of a substrate.

On the one hand, the aforementioned use is a method for carrying out a chemical reaction using
at least one platinum(IV) complex according to general formula

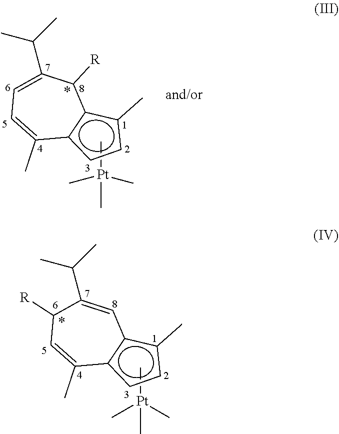

and/or
according to formula

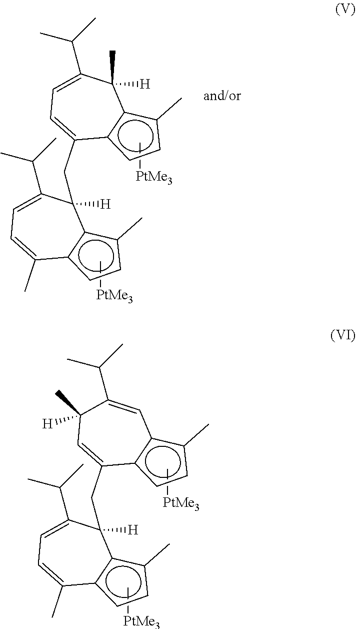

or
at least one solution or suspension comprising a platinum(IV) complex according to general formula

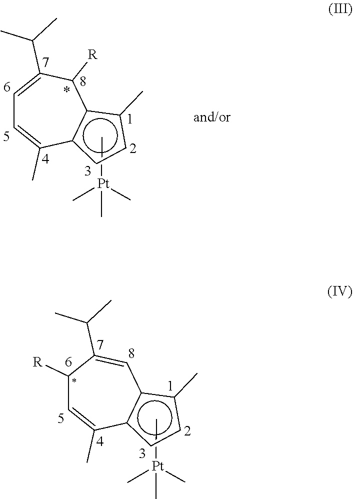

and a solvent that is in particular miscible with or identical to the solvent S_{P}
and/or
according to formula

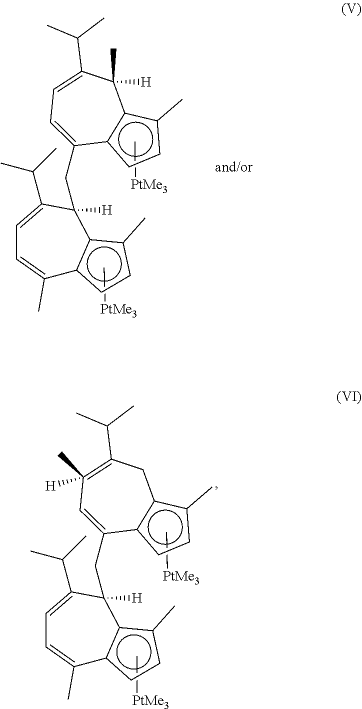

and a solvent SD that comprises or is a nonpolar aprotic or a polar aprotic solvent, in particular is miscible with or identical to the solvent S_{P}, wherein
R is in each case selected from the group consisting of primary, secondary, tertiary alkyl, alkenyl and alkynyl radicals having 1 to 10 carbon atoms, cyclic alkyl radicals having 3 to 10 carbon atoms, a benzyl radical, mononuclear aryl radicals, polynuclear aryl radicals, mononuclear heteroaryl radicals and polynuclear heteroaryl radicals,
   according to an embodiment of the platinum(IV) complexes described above
or
   according to an embodiment of the solutions or suspensions described above, comprising such a platinum(IV) complex,
or
   obtained or obtainable according to a method for producing such platinum(IV) complexes or solutions or suspensions according to any one of the embodiments described further above,
comprising the steps of:
A) providing the at least one platinum(IV) complex according to general formula III and/or IV and/or according to formula V and/or VI
or
the at least one solution or suspension, comprising a platinum(IV) complex according to general formula III and/or IV and a solvent that is in particular miscible with or identical to the solvent S_{P}, and/or comprising a platinum(IV) complex according to formula V and/or VI and a solvent SD that comprises or is a nonpolar aprotic or a polar aprotic solvent, in particular is miscible with or identical to the solvent S_{P}
and
B) carrying out the chemical reaction using the at least one platinum(IV) complex according to general formula III and/or IV and/or according to formula V and/or VI as a precatalyst and/or as a catalyst.

In this case, the general formulae III and IV each comprise both the monomers and any oligomers, in particular dimers, and solvent adducts. Formulae V and VI each also comprise solvent adducts.

In addition, the compounds according to general formulae III and IV to be used here, are in particular each present as a diastereomer mixture. In other words: In the case of the platinum(IV) complex according to formula III, the carbon atom C8 is a stereocenter. In the case of the platinum(IV) complex according to formula IV, the carbon atom C6 is a stereocenter. Alternatively, a diastereomer mixture comprising, in particular consisting of, the diastereomer mixture of the compound according to formula III and the diastereomer mixture of the compound according to formula IV, can be present in particular as a function of the selected substituent R. In each of the aforementioned cases, each diastereomer is present as an enantiomer mixture, wherein the diastereomers can be present independently of one another as a racemate.

The platinum(IV) complexes according to general formulae III, IV, V and VI are usually obtained solvent-free, i.e., not as solvent adducts, or are generally present in a solvent-free form. However, by means of the methods described further above for producing such platinum(IV) complexes, in particular according to formula III and formula IV, solvent adducts of these metal complexes can also be obtained. In the case of such an adduct, the solvent is in particular identical to the solvent S_{P} used in the context of the method described further above if the solvent S_{P} is an alkoxyalkane or the solvent Se contains at least one alkoxyalkane.

In particular with respect to use as platinum precursor compounds in vapor deposition processes, it is advantageous if the platinum(IV) complexes according to general formula III and/or IV and/or V and/or VI are present in particular free of alkoxyalkane.

Chemical reactions that can be carried out using the at least one platinum(IV) complex according to general formula III and/or according to general formula IV and/or according to general formula IV and/or according to general formula V as a precatalyst and/or as a catalyst include, in a non-limiting manner, light-induced platinum-catalyzed hydrosilylation reactions, the hydrogenation of unsaturated compounds, and polymerization reactions in which the catalyst activation is effected by ultraviolet or visible radiation.

On the other hand, the aforementioned use is a method for producing
i. at least one layer consisting of platinum
or
ii. at least one layer containing platinum on at least one surface of a substrate using
   at least one platinum(IV) complex according to general formula

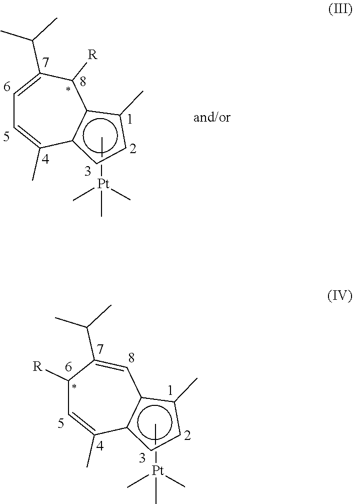

or
   Q at least one solution or suspension comprising a platinum(IV) complex according to general formula

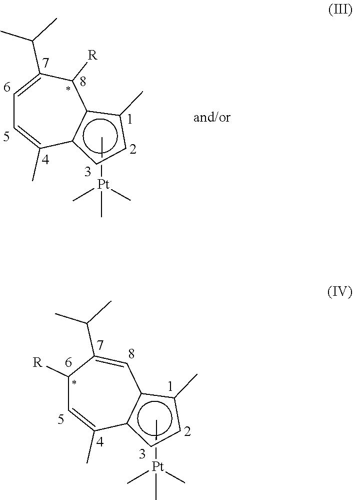

and a solvent that is in particular miscible with or identical to the solvent S_{P},
wherein
R is in each case selected from the group consisting of primary, secondary, tertiary alkyl, alkenyl and alkynyl radicals having 1 to 10 carbon atoms, cyclic alkyl radicals having 3 to 10 carbon atoms, a benzyl radical, mononuclear aryl radicals, polynuclear aryl radicals, mononuclear heteroaryl radicals and polynuclear heteroaryl radicals,
   according to an embodiment of the platinum(IV) complexes described above
or
   according to an embodiment of the solutions or suspensions described above, comprising such a platinum(IV) complex,
or
   obtained or obtainable according to a method for producing such platinum(IV) complexes or such solutions or suspensions according to any one of the embodiments described further above,
comprising the steps of:
A) providing
the at least one platinum(IV) complex according to general formula III and/or IV
or
the at least one solution or suspension, comprising a platinum(IV) complex according to general formula III and/or IV and a solvent that is in particular miscible with or identical to the solvent S_{P}
and
B) depositing
i. the at least one layer consisting of platinum
or
ii. the at least one layer containing platinum
on the at least one surface of the substrate using the at least one platinum(IV) complex according to general formula III and/or IV as precursor compound.

In the uses described herein or the method claimed herein for carrying out a chemical reaction or for producing at least one platinum layer or at least one layer containing platinum, the platinum(IV) complexes can be used as solids, liquids, solutions or suspensions according to an embodiment of the platinum(IV) complexes described further above. Alternatively, the platinum(IV) complexes or solutions or suspensions comprising a platinum(IV) complex and at least one solvent which is in particular miscible with or identical to the solvent S_{P}, for example S_{L}, in each case obtained or obtainable according to a method for producing such platinum(IV) complexes or solutions or suspensions according to one of the embodiments described further above, can be used as a solid, liquid, solution or suspension. Due to their high purity, the aforementioned Pt(IV) complex compounds according to formulae III, IV, V and VI, in particular according to formulae III and IV, are suitable for use both as precatalysts and/or as catalysts in a plurality of reactions that are catalyzed by platinum, and as platinum precursor compounds in vapor deposition processes, in particular low-temperature vapor deposition processes. Some of these platinum(IV) complexes are advantageously present in liquid form, not only in high purity but also at room temperature.

A definition of the term “platinum(IV) complex in high purity” is given further above.

The platinum(IV) complex according to the general formulae III and IV each have an organo-dihydroguaiazulenyl anion or R-dihydroguaiazulenyl anion (GuaR)¹⁻ which carries an organyl radical R in the 8 position or in the 6 position of the guaiazulene skeleton in addition to an H atom. The R-dihydroguaiazulenyl anion (GuaR)¹⁻ can therefore be a 7-iso-propyl-1,4-dimethyl-8-R-dihydroazulenyl anion or 8-R-dihydroguaiazulenyl anion (Gua-8-R)¹⁻ according to formula III or a 7-iso-propyl-1,4-dimethyl-6-R-dihydroazulenyl anion or 6-R-dihydroguaiazulenyl anion (Gua-6-R)¹⁻ according to formula IV. In other words: There is an RCH group in the C8 position or in the C6 position of the guaiazulene skeleton. The platinum(IV) complexes according to formula V and formula VI each have two 4,8-linked organo-dihydroguaiazulenyl radical anions. In this case, the radical anion involved in the linkage via its C4 atom in the 8 position or in the 6 position of the guaiazulene skeleton carries, in addition to an H atom, an organyl radical R, in particular a methyl radical. As a result of the addition of an organyl anion (R)¹⁻, the aromaticity is limited to the five-membered ring, wherein the blue color typical for azulene and derivatives thereof is generally lost. The organo-dihydroazulenyl ligand anion (GuaR)¹⁻ is a derivative of the cyclopentadienyl anion or a cyclopentadienyl-like monoanion.

Owing to the presence of an R-dihydroguaiazulenyl anion, i.e., of a cyclopentadienyl-like monoanion, the compounds according to general formulae III and IV are suitable in particular as precatalysts and/or catalysts for chemical reactions in which platinum(IV) complexes having cyclopentadienyl ligands are otherwise used. The same applies to the platinum(IV) complexes according to formula V and formula VI, which each have two 4,8-linked organo-dihydroguaiazulenyl radical anions. It is particularly advantageous that the provision of an R-dihydroguaiazulenyl ligand is less labor-intensive and time-consuming compared to the provision of the cyclopentadienyl ligand. In particular, the compounds used as reactants herein, which comprise cyclopentadienyl-like ligands, can be prepared using inexpensive renewable raw materials. This is because guaiazulene is partially accessible synthetically, namely starting from the natural material guaiacol and other azulene formers by simple dehydration and dehydrogenation (T. Shono, N. Kise, T. Fujimoto, N. Tominaga, H. Morita, J. Org. Chem. 1992, 57, 26, 7175-7187; CH 314 487 A (B. Joos) Jan. 29, 1953). As a result, the synthesis effort and the production costs for the platinum(IV) complexes claimed herein, as well as for solutions or suspensions comprising at least one such Pt(IV) compound and a solvent, which is in particular miscible with or identical to the solvent S_{P}, are also lower than for analogous platinum(IV)-Cp complexes. Consequently, the platinum(IV) complexes described herein and their solutions and suspensions represent an alternative to platinum(IV)-Cp complexes, particularly with respect to an industrial application.

Guaiazulene is a natural substance that contains chamomile oil and other essential oils and is therefore advantageously inexpensive to obtain in large quantities. It can be produced synthetically from the guaiacol of guaiac wood oil (guaiac resin). Guaiazulene is an intensely blue substance with anti-inflammatory properties.

The platinum(IV) complexes according to formula III and/or according to formula IV and/or according to formula V and/or according to formula VI, in particular the platinum(IV) complexes according to formula III and/or formula IV, and the solutions or suspensions thereof in a solvent which is in particular miscible with or identical to the solvent S_{P}, for example S_{L}, can be obtained advantageously in a simple, reproducible and comparatively cost-effective manner, in a high purity of 97%, advantageously higher than 97%, in particular higher than 98% or 99%, and in a good yield of typically ≥60%, advantageously ≥70%, and in a good space-time yield, in particular by means of one of the methods described further above for producing such platinum(IV) complexes, solutions and suspensions. In general, the end product may still contain residues of solvents or for example impurities from the reactants. It is known to a person skilled in the art that the content of impurities, for example solvents, can be determined by gas chromatography (GC) methods, optionally with mass spectrometry coupling (GC-MS).

Definitions of the terms “platinum(IV) complex in a high purity” and “space-time yield” are already given above.

An example of a platinum(IV) complex used in the uses claimed herein or the methods claimed herein for carrying out a chemical reaction or for producing at least one layer consisting of platinum or at least one layer containing platinum is the semi-sandwich complex [PtMe₃(GuaMe)]. As already explained above, said semi-sandwich complex is advantageously present in an isolated form as a diastereomer mixture that is liquid at room temperature, consisting of two or four diastereomeric enantiomer pairs. Each of the diastereomers may optionally be present as a racemate.

The isolated platinum(IV) complex [PtMe₃(GuaMe)] is easier to handle than previously known precatalysts containing cyclopentadienyl anions and frequently present in a waxy form, such as [PtMe₃(MeCp)] due to the fact that it is present in the form of an oil. As a platinum(IV) compound in liquid form at room temperature, [PtMe₃(GuaMe)] is also particularly suitable as a platinum precursor compound for vapor deposition processes, in particular low-temperature vapor deposition processes.

The platinum(IV) compound [PtMe₃(GuaMe)] also shows absorption in the VIS range. This is a further advantage in the context of light-induced platinum-catalyzed reactions, for example hydrosilylation reactions. This is because the use of UV/VIS light is regularly provided, as a result of which special safety measures are usually required in order to reduce the risk of skin cancer. Such safety measures are not absolutely necessary when [PtMe₃(GuaMe)] is used.

Furthermore, it is advantageous that the monomeric compound [PtMe₃(GuaMe)] can be stored at room temperature for several months in the absence of light. In the meantime, no decomposition reactions, oligomerization or polymerization are observed. It is also advantageous that the complex [PtMe₃(GuaMe)] has a relatively high thermal stability, i.e., it can also be used at elevated reaction or deposition temperatures. According to thermogravimetric analysis (TGA), 3% degradation occurs at a temperature of 180° C.

In step A) of the method described herein for carrying out a chemical reaction or for producing at least one layer consisting of platinum or at least one layer containing platinum, the provision of a platinum(IV) complex or a plurality of platinum(IV) complexes can be provided in each case. In an embodiment of the respective method, it is provided that in step A) at least one platinum(IV) complex is provided as a solid, as a liquid or as a solution or suspension comprising a platinum(IV) complex. Alternatively or additionally, a plurality of platinum(IV) complexes can be provided independently of one another as separate solids or as solid mixtures or as separate liquids or as liquid mixtures or as separate solutions or suspensions comprising a platinum(IV) complex, or as solutions or suspensions comprising a plurality of platinum(IV) complexes.

At this point as well as in the following, the specification of an exact stoichiometry of the depositable platinum-containing layers or films has been dispensed with. The term “layer” is synonymous with the expression “film” and does not make any statement regarding the layer thickness or the film thickness. In addition, according to the present invention, a platinum layer or a layer containing platinum can contain nanoparticles of a metal Q, in particular platinum nanoparticles, or nanoparticles of different metals Q or nanoparticles, each comprising a plurality of metals Q, or can consist of such nanoparticles.

Due to their high degree of purity, the platinum(IV) complexes used are particularly suitable as precursor compounds for producing high-quality platinum layers or platinum-containing layers on a surface of a substrate. This is due in particular to its production according to a method according to one of the embodiments described above, namely using alkali metal R-dihydroguaiazulenides according to formula I and/or formula II. In addition, the platinum(IV) complexes or solutions or suspensions that are to be provided according to step A) comprising such complexes are relatively simple, reproducible, sustainable and cost-effective to prepare according to a method for producing such compounds or solutions or suspensions according to one of the embodiments described above. The latter allows them to be used on an industrial scale.

In one embodiment of the uses claimed herein or the method claimed herein for carrying out a chemical reaction or for producing at least one layer consisting of platinum or containing platinum, R is selected from the group consisting of primary, secondary, tertiary alkyl, alkenyl and alkynyl radicals having 1 to 10 carbon atoms, cyclic alkyl radicals having 3 to 10 carbon atoms, a benzyl radical, mononuclear aryl radicals, polynuclear aryl radicals, mononuclear heteroaryl radicals and polynuclear heteroaryl radicals. The radical R may therefore also be primary, secondary, tertiary alkyl, alkenyl and alkynyl radicals having 2, 3, 4, 5, 6, 7, 8 or 9 carbon atoms, and cyclic alkyl radicals having 4, 5, 6, 7, 8 or 9 carbon atoms. According to another embodiment of the uses or methods claimed herein, R is selected from the group consisting of primary, secondary, tertiary alkyl, alkenyl and alkynyl radicals having 1 to 6 carbon atoms, cyclic alkyl radicals having 3 to 6 carbon atoms, a benzyl radical, mononuclear aryl radicals, polynuclear aryl radicals, mononuclear heteroaryl radicals and polynuclear heteroaryl radicals. The radical R may therefore also be primary, secondary, tertiary alkyl, alkenyl and alkynyl radicals having 2, 3, 4 or 5 carbon atoms, and cyclic alkyl radicals having 4 or 5 carbon atoms. In yet another variant of the uses or methods claimed herein, R is selected from the group consisting of Me, Et, n-Pr, i-Pr, n-Bu, i-Bu, s-Bu, t-Bu, n-pentyl, 2-pentyl, 3-pentyl, 2-methylbutyl, 3-methylbutyl, 3-methylbut-2-yl, 2-methylbut-2-yl, 2,2-dimethylpropyl, cyclopentyl, cyclohexyl, phenyl, tolyl, benzyl and cumyl, and isomers thereof. According to another advantageous embodiment, R is selected from the group consisting of Me, Et, n-Pr, i-Pr, n-Bu, i-Bu, s-Bu, t-Bu, cyclohexyl, phenyl, tolyl, benzyl and cumyl, and isomers thereof; R is particularly advantageously selected from the group consisting of Me, Et, n-Pr, i-Pr, n-Bu, i-Bu, s-Bu, t-Bu, in particular R=Me.

In a further embodiment of the uses described herein or the method claimed herein for carrying out a chemical reaction or for producing at least one layer consisting of platinum or containing platinum, the platinum(IV) complex is selected from the group consisting of [PtMe₃(GuaMe)], [PtMe₃(GuaEt)], [PtMe₃(GuanPr)], [PtMe₃(GuaiPr)], [PtMe₃(GuanBu)], [PtMe₃(GuaiBu)], [PtMe₃(GuatBu)], [PtMe₃(GuasBu)] and [PtMe₃(GuaPh)]. In particular, the platinum(IV) complex is [PtMe₃(GuaMe)]. As already explained above, the semi-sandwich complex [PtMe₃(GuaMe)] is advantageously present in an isolated form as a diastereomer mixture that is liquid at room temperature, consisting of two or four diastereomeric enantiomer pairs, wherein each of the diastereomers is optionally present as a racemate.

In another embodiment of the method claimed herein for producing a platinum layer or a platinum-containing layer, the deposition of the platinum layer or of the platinum-containing layer in step B) takes place by means of a vapor deposition process, in particular a low-temperature vapor deposition process. The platinum layer or the platinum-containing layer is advantageously deposited by means of an ALD process or an MOCVD process, in particular by means of an MOVPE process. Alternatively, a sol-gel process can be used, wherein the sol can be deposited on one or more surfaces of the substrate by means of spin coating or dip coating, for example.

A further variant of the use described herein or the claimed method provides for the sequential deposition of a plurality of platinum layers and/or platinum-containing layers on the surface of the substrate. In this case, step B) is repeated, wherein the respective platinum-containing layers and/or platinum layers are deposited one after the other. The first layer is deposited directly on the surface of the substrate, while subsequent layers are deposited on the surface of the previously deposited layer.

The substrate may, for example, comprise one non-metal or a plurality of base metals or be manufactured from one non-metal or a plurality of base metals. Alternatively or additionally, the substrate may comprise one non-metallic material or a plurality of non-metallic materials or consist entirely of one non-metallic material or a plurality of such non-metallic materials. Corundum foils or thin metallic foils can, for example, be used as the substrate. The substrate itself can be part of a component. In one embodiment of the aforementioned use of a platinum(IV) complex as precursor compound for producing a platinum layer or a platinum-containing layer, or in one embodiment of the method for producing a platinum layer or a platinum-containing layer on a surface of a substrate, the substrate is a wafer. The wafer may comprise silicon, silicon carbide, germanium, gallium arsenide, indium phosphide, a glass, such as SiO₂, and/or a plastic, such as silicone, or consist entirely of one or more such materials. The wafer can also have one or more wafer layers, each having one surface. The production of the platinum layer or of the platinum-containing layer may be provided on the surface of one or more wafer layers.

Due to the very high degree of purity of the platinum layer or of the platinum-containing layer, substrates obtained or obtainable by means of the use claimed herein or the method described herein and comprising a platinum layer or a platinum-containing layer, optionally comprising metal nanoparticles or consisting of metal nanoparticles, can be used particularly well for the production of an electronic component, in particular of an electronic semiconductor component, or of a redox-active electrode for a fuel cell. In the latter case, the platinum layer or the platinum-containing layer functions as a catalytic layer.

The object is also achieved by a substrate comprising
i. at least one layer consisting of platinum
or
ii. at least one layer containing platinum
on at least one surface,
wherein the at least one layer consisting of platinum or the at least one layer containing platinum is producible or produced using
a platinum(IV) complex according to general formula III and/or IV
or
a solution or suspension comprising such a platinum(IV) complex and a solvent that is in particular miscible with or identical to the solvent S_{P}
   according to an embodiment of the platinum(IV) complexes described above
or
   according to an embodiment of the solutions or suspensions described above, comprising such a platinum(IV) complex,
or
   obtained or obtainable according to a method for producing such platinum(IV) complexes or such solutions or suspensions comprising such a platinum(IV) complex according to any of the embodiments described further above.

With respect to a selection of usable platinum(IV) complexes according to general formula III and/or general formula IV and of solutions or suspensions comprising such a platinum(IV) complex and a solvent which is in particular miscible with or identical to the solvent S_{P}, reference is made to the statements regarding the method described further above for producing such a substrate.

A definition of the term “miscible” has already been given above.

With regard to the advantages of such a substrate, reference is made to the advantages mentioned for the method described further above for producing such a substrate.

The substrate described herein can be obtained in particular by means of the method described further above for producing at least one layer consisting of platinum or at least one layer containing platinum on at least one surface of a substrate.

Furthermore, the object is achieved by a crosslinkable silicone composition comprising
i. at least one compound selected from the group consisting of type (a) compounds, type (b) compounds and type (c) compounds, wherein
   type (a) compounds are organic compounds and organosilicon compounds, each comprising at least two radicals having aliphatic carbon-carbon multi-bonds,
   type (b) compounds are organosilicon compounds, each comprising at least two Si-bonded hydrogen atoms, and
   type (c) compounds are organosilicon compounds, each comprising SiC-bonded radicals having aliphatic carbon-carbon multi-bonds and Si-bonded hydrogen atoms,
wherein the silicone composition contains at least one compound having aliphatic carbon-carbon multi-bonds and at least one compound having Si-bonded hydrogen atoms,
and
ii. at least one platinum(IV) complex according to any of the formulae III, IV, V and VI according to any of the embodiments described further above.

The crosslinkable silicone composition can be easily obtained by mixing the respectively desired compounds, wherein the desired compounds can be mixed with one another in any sequence.

Silicone elastomers can be produced by means of the crosslinkable, in particular addition-crosslinkable, silicone composition claimed herein. In such crosslinkable silicone compositions, the crosslinking process generally takes place via a hydrosilylation reaction, in which platinum or another metal from the platinum group is usually used as a catalyst. In the reaction proceeding by catalysis, aliphatic unsaturated groups are reacted with Si-bonded hydrogen in order to convert the crosslinkable silicone composition to the elastomeric state via the structure of a network. According to the prior art, the catalysts used are usually activated thermally or by UV/VIS radiation. The former procedure is usually relatively costly. Although UV/VIS radiation is more cost-effective, it generally requires special safety measures to reduce the risk of skin cancer. Such safety measures are not absolutely necessary when using a crosslinkable, in particular addition-crosslinkable, silicone composition described herein comprising at least one platinum(IV) complex according to any of the formulae III, IV, V and VI according to any of the embodiments described further above, in particular [PtMe₃(GuaMe)]. This is because the platinum(IV) complexes described further above, each of which comprises at least one organo-dihydroguaiazulenyl ligand (GuaR)¹⁻ or two 4,8-linked organo-dihydroguaiazulenyl radical anions, exhibit absorption in the visible range and can therefore be activated by means of visible radiation.

In particular, platinum(IV) complexes according to formula III and/or according to formula IV and the solutions or suspensions thereof in a solvent, which is in particular miscible with or identical to the solvent S_{P}, for example S_{L}, can be obtained, advantageously in a simple, reproducible and comparatively cost-effective manner, in a high purity of 97%, advantageously higher than 97%, in particular higher than 98% or 99%, and in a good yield of typically ≥60%, advantageously ≥70%, and in a good space-time yield, in particular by means of the method described further above for the preparation of such platinum(IV) complexes, solutions and suspensions. In general, the end product may still contain residues of solvents or for example impurities from the reactants. It is known to a person skilled in the art that the content of impurities, for example solvents, can be determined by gas chromatography (GC) methods, optionally with mass spectrometry coupling (GC-MS).

Definitions of the terms “platinum(IV) complex in a high purity” and “space-time yield” are already given above.

Finally, the crosslinkable silicone compositions claimed herein can advantageously be obtained in high purity in a simple, reproducible and comparatively cost-effective manner. It is also advantageous that the use of these compositions for producing silicone elastomers can be realized in a particularly simple, safe and cost-effective manner.

According to an embodiment of the crosslinkable silicone composition claimed herein, the silicone composition comprises
i. at least one type (a) and type (b) compound
or
ii. at least one type (c) compound
or
iii. at least one type (a) and type (c) compound
or
iv. at least one type (b) and type (c) compound
or
v. at least one type (a), type (b) and type (c) compound.

Other characteristics, details, and advantages of the invention follow from the exact wording of the claims, as well as from the following description of the embodiment examples based upon the illustrations. In the figures:

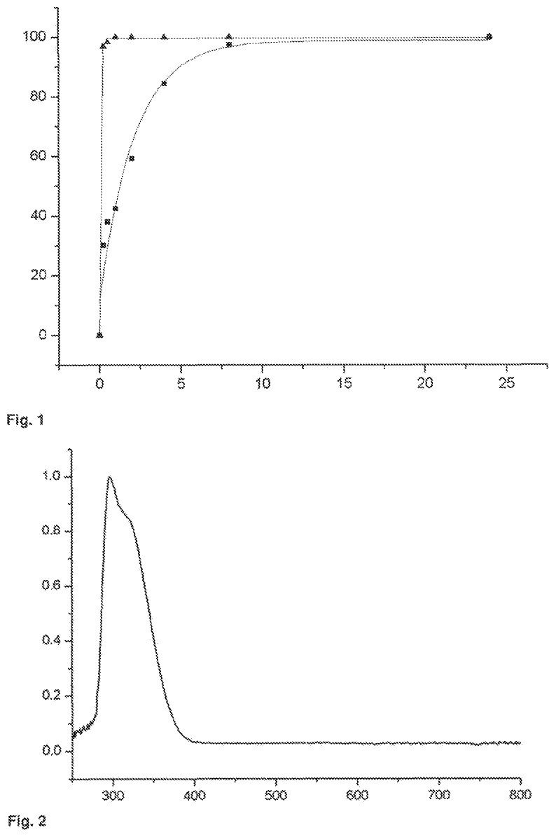

FIG. 1 shows a diagram relating to the conversion of the hydrosilylation reaction of 1-octene with pentamethyldisiloxane using 5 ppm of [PtMe₃(GuaMe)] (triangles) or 5 ppm of [PtMe₃(CpMe)] (squares) as a precatalyst,

FIG. 2 shows UV/VIS spectrum of [PtMe₃(GuaMe)], taken up in cyclohexane (10⁻⁵ mol/L).

During the hydrosilylation reaction of 1-octene with pentamethyldisiloxane using 5 ppm of [PtMe₃(GuaMe)] as a precatalyst (see example 4, NMR experiments with 5 ppm of Pt), ¹H-NMR spectra (C₆D₆, 298 K, 300 MHz) were recorded at predefined time intervals. A first ¹H-NMR spectrum was recorded at time 0 h, i.e., before starting the hydrosilylation reaction. After about 0.25 h, 0.5 h, 1 h, 2 h, 4 h, 8 h and 24 h, further ¹H-NMR spectra were recorded.

The reaction equation for the hydrosilylation of 1-octene is as follows:

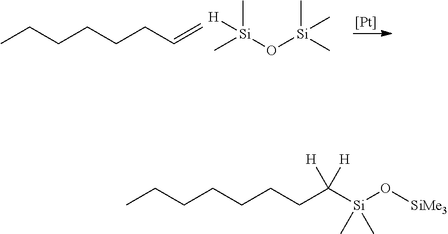

The conversion was in each case based on the CH₂ group of the product (highlighted in gray in the product molecule) with a shift of 0.60 ppm. All ¹H-NMR spectra were taken into account in the calculation of the conversions. Table 1 shows the calculated conversions of the hydrosilylation reaction of 1-octene with pentamethyldisiloxane using 5 ppm of [PtMe₃(GuaMe)] as a precatalyst (see column 2). Under identical conditions, the hydrosilylation reaction of 1-octene with pentamethyldisiloxane was carried out using 5 ppm of [PtMe₃(CpMe)]. The calculated conversions are also indicated in Table 1 (see column 3).

TABLE 1

Conversion in %

| Time in h | [PtMe₃(GuaMe)] | [PtMe₃(CpMe)] |
|---|---|---|
| 0 | 0 | 0 |
| 0.25 | 96.7 | 30.1 |
| 0.5 | 98.2 | 37.7 |
| 1 | 100 | 42.7 |
| 2 | 100 | 60.2 |
| 4 | 100 | 85.4 |
| 8 | 100 | 98.3 |
| 24 | 100 | 99.7 |

The graphical evaluation of the results listed in Table 1 is shown in FIG. 1. The time in hours (h) is plotted on the abscissa and the conversion in percent (%) is plotted on the ordinate. The triangular data points indicate the conversions calculated as a precatalyst when using [PtMe₃(GuaMe)]. The square data points indicate the calculated conversions that were achieved with [PtMe₃(CpMe)] as a precatalyst.

It can be seen that the hydrosilylation reaction using 5 ppm of [PtMe₃(GuaMe)] is already almost complete after approximately 1 h. Consequently, a very satisfactory reaction rate is achieved that, under identical conditions, is in particular higher than that of the Pt(IV) complex [PtMe₃(CpMe)] known as a hydrosilylation catalyst. In other words: The precatalyst [PtMe₃(GuaMe)] has a higher activity than [PtMe₃(CpMe)].

Advantageously, the Pt(IV) compound [PtMe₃(GuaMe)] is present as a liquid and exhibits absorption in the visible range. The UV/VIS spectrum of [PtMe₃(GuaMe)] is shown in FIG. 2. The wavelength in nm is plotted on the abscissa and the absorption is plotted in arbitrary units (a. u.) on the ordinate. A shoulder is observed at >340 nm. This is caused by the two olefinic double bonds of the seven-membered ring, which are conjugated with the aromatic system of the five-membered ring. Contamination by neutral guaiazulene, optionally formed by abstraction of an H atom in the 8 position of the dihydroguaiazulenide ligand, is not detectable in the visible range (blue-green) of the spectrum shown in FIG. 2.

The fact that the complex [PtMe₃(GuaMe)] exhibits absorption in the visible range is a further advantage of this Pt(IV) compound used here as a precatalyst. This is because, in the case of light-induced platinum-catalyzed hydrosilylation reactions, the use of UV/VIS light is regularly provided, as a result of which special safety measures are usually required in order to reduce the risk of skin cancer. Such safety measures are not absolutely necessary when [PtMe₃(GuaMe)] is used.

According to conventional wisdom, photolysis of a known precatalyst comprising a cyclopentadienyl anion, for example [PtMe₃(Cp)] and [PtMe₃(CpMe)], in the presence of a silane, for example pentamethyldisiloxane, results in the formation of a platinum colloid as an active hydrosilylation catalyst. (L. D. Boardman, Organometallics 1992, 11, 4194-4201) For the hydrosilylation reaction of 1-octene with pentamethyldisiloxane (equimolar mixture), Boardman specifies a precatalyst concentration of 10 ppm of platinum as [PtMe₃(Cp)].

When using the precatalyst [PtMe₃(GuaMe)] shown for the first time in the context of the present invention, a complete conversion of the substrates 1-octene and pentamethyldisiloxane is already observed, namely already after about 1 h, at 50% of the catalyst concentration selected in the literature, i.e., at a precatalyst concentration of 5 ppm of platinum as [PtMe₃(GuaMe)].

A further advantage of the compound [PtMe₃(GuaMe)] used in the present case as a precatalyst over Pt(IV) compounds containing cyclopentadienyl anions is that the guaiazulene required for the production thereof is synthesized using renewable raw materials instead of crude oil. As a result, the preparation of the Pt(IV) complex [PtMe₃(GuaMe)] can be realized in a comparatively sustainable, simple and cost-effective manner.

Consequently, the semi-sandwich complex [PtMe₃(GuaMe)] used in the context of the present invention for the hydrosilylation reaction of 1-octene with pentamethyldisiloxane is a relatively sustainable and cost-effective alternative to known hydrosilylation precatalysts such as [PtMe₃(Cp)] and [PtMe₃(CpMe)].

### Protocols for the Synthesis of Li(GuaMe) and [PtMe₃(GuaMe)]

(GuaMe)⁻=C₁₅H₁₉⁻
=7-iso-propyl-1,4-dimethyl-8-methyl-dihydroazulenyl-anion (Gua-8-Me)¹⁻ or
   7-iso-propyl-1,4-dimethyl-6-methyl-dihydroazulenyl-anion (Gua-6-Me)¹⁻

### Materials and Methods

All reactions were carried out under standard inert gas conditions. The solvents and reagents used were purified and dried according to standard procedures.

All nuclear magnetic resonance spectroscopic measurements were carried out on a Bruker AV II 300, Bruker AV II HD 300, DRX 400 or AV III 500 device. ¹³C-NMR spectra were measured in a standard ¹H broadband decoupled manner at 300 K. ¹H and ¹³C-NMR spectra were calibrated to the corresponding residual proton signal of the solvent as an internal standard: ¹H: DMSO[d₆]: 2.50 ppm, C₆D₆: 7.16 ppm (s); ¹³C: DMSO[d₆]: 39.52 ppm; C₆D₆: 128.0 ppm (t). The chemical shifts are indicated in ppm and refer to the δ scale. All signals are provided with the following abbreviations according to their splitting pattern: s (singlet), d (doublet), dd (double doublet), q (quartet) or sept (septet).

High-resolution LIFDI mass spectra were obtained using an AccuTOF-GCv-TOF mass spectrometer (JEOL).

UV/VIS spectra were recorded with an Avantes AvaSpec-2048 spectrophotometer in 10 mm cuvettes in cyclohexane at 10 μM concentrations at a scan rate of 600 nm/min at room temperature.

Thermogravimetric studies were performed with a DSC-TGA 3 (Mettler Toledo) in a glove box. The samples were heated in aluminum crucibles at a heating rate of 10 K/min to the final temperature. The decomposition temperatures were determined using the data from the DSC TGA. The evaluation of the spectra obtained was carried out with STARe software made by Mettler Toledo.

### Example 1: Preparation of Li(GuaMe)

The Li(GuaMe) reactant was determined on the basis of the synthesis described by Edelmann and his collaborators for lithium-7-iso-propyl-1,4,8-trimethyl-dihydroguaiazulenide (J. Richter, P. Liebing, F. T. Edelmann, Inorg. Chim. Acta 2018, 475, 18-27):

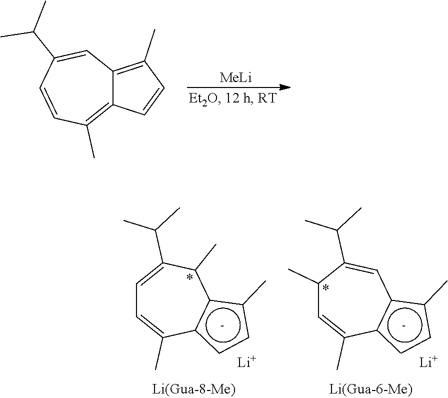

Guaiazulene (4.00 g, 20.2 mmol) in 40 mL diethyl ether was admixed with MeLi (1.59 M in diethyl ether, 12.68 mL) at 0° C. The mixture was warmed to room temperature and stirred for a further 12 h. During this time, the blue color disappeared and a brown suspension formed. The precipitated product was isolated by filtration, washed with diethyl ether (3×20 mL) and dried in vacuo to give Li(GuaMe) as an off-white, highly air- and moisture-sensitive solid (3.35 g, 15.2 mmol, 75%).

¹H-NMR (300.1 MHz, DMSO-d₆): δδ=5.42 (d, ³J_{HH}=3.5 Hz, 1H), 5.37 (d, ³J_{HH}=3.5 Hz, 1H), 5.33 (d, ³J_{HH}=7.0 Hz, 1H), 4.84 (d, ³J_{HH}=6.8 Hz, 1H), 3.41 (q, ³J_{HH}=7.2 Hz, 1H), 2.34 (sept, ³J_{HH}=6.7 Hz, 1H), 2.02 (s, 3H), 1.99 (s, 3H), 1.04 (d, ³J_{HH}=6.7 Hz, 3H), 1.00 (d, ³J_{HH}=6.7 Hz, 3H), 0.70 (d, ³J_{HH}=6.9 Hz, 3H) ppm; ¹³C-NMR (300.1 MHz, DMSO-d₆): δ=140.1 (s, 1C), 135.4 (s, 1C), 121.3 (s, 1C), 120.3 (s, 1C), 117.7 (s, 1C), 108.7 (s, 1C), 106.4 (s, 1C), 106.2 (s, 1C), 100.6 (s, 1C), 37.0 (s, 1C), 33.5 (s, 1C), 24.2 (s, 1C), 23.2 (s, 1C), 22.7 (s, 1C), 20.0 (s, 1C), 13.9 (s, 1C).

### Note:

The Li(GuaMe) compound was obtained as a mixture of the two regioisomers lithium-7-iso-propyl-1,4,8-trimethyl-dihydroguaiazulenide (Li(Gua-8-Me)) and lithium-7-iso-propyl-1,4,6-trimethyl-dihydroguaiazulenide (Li(Gua-6-Me)). According to ¹H-NMR spectroscopic analysis, the isolated isomer mixture consisted of 12 mol % to 15 mol % of Li(Gua-6-Me) and 85 mol % to 88 mol % of Li(Gua-8-Me).

### Example 2: Preparation of [PtMe₃(GuaMe)]

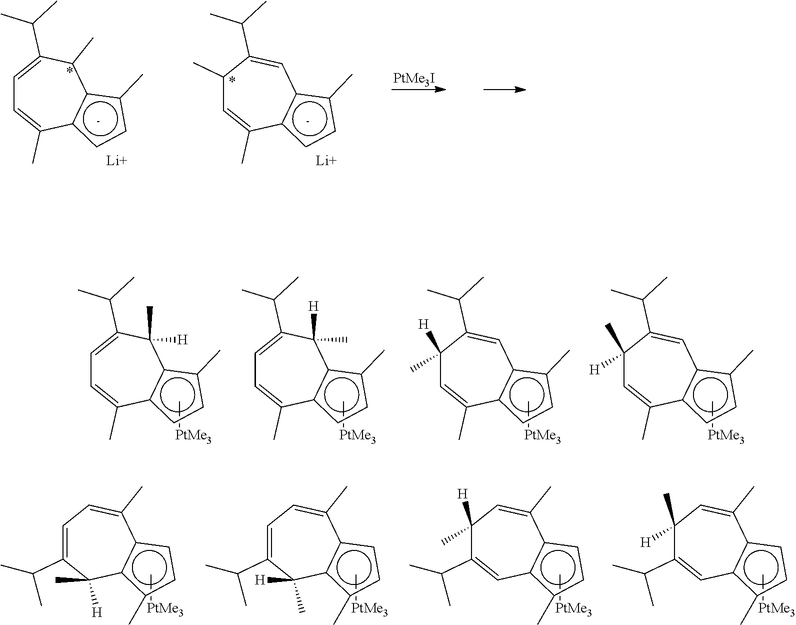

Li(GuaMe) (243 mg, 1.10 mmol) and [PtMe₃l]₄ (405 mg, 0.28 mmol) were suspended in diethyl ether (20 mL), and the suspension was stirred at 40° C. for 30 min, then at room temperature for 4 h. A yellow solution was obtained. The solvent was removed in vacuo. The oily residue was taken up in pentane (40 mL), and inorganic salts and any unreacted reactants were separated off by filtration. The solvent of the filtrate was removed in vacuo, and [PtMe₃(GuaMe)] was obtained as a yellow green oil in a good yield (386 mg, 0.85 mmol, 77%). The ¹H-NMR spectrum shows a d.r. of 68% (main diastereomer, fraction 2): 32% (secondary diastereomer, fraction 1). The compound can be condensed, for example, onto a sublimation finger (acetone/dry ice, −78° C.). The product can be further purified by means of column chromatography (silica or Al₂O₃ (neutral), hexane).

Fraction 1 ([PtMe₃(Gua-8-endo-Me)], secondary diastereomer): ¹H-NMR (300.1 MHz, DMSO-d₆): δ=6.03 (dd, ³J_{HH}=2.8 Hz, 1H), 5.48 (d, ³J_{HH}=7.1 Hz, 1H), 5.43 (d, ³J_{HH}=2.7 Hz, 1H), 5.38 (d, ³J_{HH}=2.6 Hz, 1H), 3.17 (q, ³J_{HH}=6.8 Hz, 1H), 2.28 (sept, ³J_{HH}=5.4 Hz, 1H), 1.93 (s, 3H), 1.92 (s, 3H), 1.03 (d, ³J_{HH}=2.5 Hz, 3H), 1.01 (d, ³J_{HH}=2.3 Hz, 3H), 0.58 (s, ²J_{PtH}=40.6 Hz, 9H) ppm; ¹³C-NMR (300.1 MHz, DMSO-d₆): δ=149.0 (s, 1C), 126.9 (s, 1C), 123.5 (s, 1C), 118.1 (s, 1C), 116.6 (s, 1C), 110.1 (s, 1C), 109.3 (s, 1C), 92.4 (s, 1C), 86.1 (s, 1C), 36.8 (s, 1C), 31.5 (s, 1C), 22.7 (s, 1C), 21.9 (s, 1C), 21.6 (s, 1C), 19.4 (s, 1C), 9.5 (s, 1C), −13.4 (s, ¹J_{PtC}=359 Hz, 3C).

Fraction 2 ([PtMe₃(Gua-8-exo-Me)], main diastereomer): ¹H-NMR (300.1 MHz, DMSO-d₆): δ=6.06 (dd, ³J_{HH}=2.6 Hz, 1H), 5.51 (d, ³J_{HH}=6.7 Hz, 1H), 5.48 (d, ³J_{HH}=2.6 Hz, 1H), 5.28 (d, ³J_{HH}=2.6 Hz, 1H), 3.36 (q, ³J_{HH}=7.0 Hz, 1H), 2.39 (sept, ³J_{HH}=6.5 Hz, 1H), 1.98 (s, 3H), 1.92 (s, 3H), 1.02 (d, ³J_{HH}=2.6 Hz, 3H), 0.99 (d, ³J_{HH}=1.9 Hz, 3H), 0.80 (s, ²J_{PtH}=40.3 Hz, 9H) ppm; ¹³C-NMR (300.1 MHz, DMSO-d₆): δ=150.8 (s, 1C), 128.9 (s, 1C), 124.9 (s, 1C), 120.0 (s, 1C), 117.9 (s, 1C), 109.9 (s, 1C), 108.7 (s, 1C), 91.3 (s, 1C), 83.2 (s, 1C), 36.4 (s, 1C), 30.9 (s, 1C), 21.7 (s, 1C), 21.6 (s, 1C), 21.5 (s, 1C), 20.2 (s, 1C), 10.1 (s, 1C), −15.28 (s, ¹J_{PtC}=359 Hz, 3C).

TGA (T_{S}=25 K, T_{E}=600 K, 10 K/min): Stages: 1, 3% degradation: 180.0° C., total mass degradation: 50.7%.

### Note:

The diastereomers that can be prepared starting from lithium-7-iso-propyl-1,4,6-trimethyl-dihydroguaiazulenide Li(Gua-6-Me) can neither be detected nor isolated by means of ¹H-NMR spectroscopy. Only thin-layer chromatography provides indications of the four possible diastereomers [PtMe₃(Gua-6-endo-Me)], [PtMe₃(Gua-6-exo-Me)], [PtMe₃(Gua-8-endo-Me)] and [PtMe₃(Gua-8-exo-Me)].

### Example 3: Preparation of a) [PtMe₃(Gua-8-Me)-CH₂-(Gua-6-Me)PtMe₃] and b) [PtMe₃(Gua-8-Me)-CH₂-(Gua-8-Me)PtMe₃]

Li(GuaMe) (243 mg, 1.10 mmol) and [PtMe₃l]₄ (405 mg, 0.28 mmol) were suspended in diethyl ether (20 mL), and the suspension was stirred at 40° C. for 30 min, then at room temperature for 4 h. A yellow solution was obtained. The solvent was removed in vacuo. The oily residue was taken up in pentane (40 mL), and inorganic salts and any unreacted reactants were separated off by filtration. The solvent of the filtrate was removed in vacuo, and [PtMe₃(GuaMe)] was obtained as a yellow green oil in a good yield (386 mg, 0.85 mmol, 77%).

The ¹H-NMR spectrum of the yellow green oil in DMSO-d₆ (for the evaluation see Example 2) shows a d.r. of 68% ([PtMe₃(Gua-8-exo-Me)]): 32% (PtMe₃(Gua-8-endo-Me)]). The two diastereomers [PtMe₃(Gua-6-endo-Me)] and [PtMe₃(Gua-6-exo-Me)] cannot be detected by means of ¹H-NMR spectroscopy.

a) [PtMe₃(Gua-8-Me)-CH₂-(Gua-6-Me)PtMe₃]

The sample examined by means of ¹H-NMR spectroscopy was stored for three days under daylight irradiation at room temperature.

As a result, crystals of the dimeric compound [PtMe₃(Gua-8-Me)-CH₂-(Gua-6-Me)PtMe₃] suitable for X-ray structure analysis were obtained. In addition, a toluene solution of a crystal was analyzed by means of mass spectrometry. A high resolution of a LIFDI (FD+) spectrum shows the associated peak at m/z=890.36474 (m/z calculated for [C₃₇H₅₆Pt₂]⁺: 890.36775), namely in accordance with the calculated isotope pattern.

b) [PtMe₃(Gua-8-Me)-CH₂-(Gua-8-Me)PtMe₃]

The yellow green oil was subjected to sublimation (acetone/dry ice, −78° C.) and column chromatography (silica or Al₂O₃ (neutral), hexane). A sample of the isolated diastereomer mixture consisting of [PtMe₃(Gua-8-exo-Me)] and [PtMe₃(Gua-8-endo-Me)] was dissolved in DMSO. The solution was stored at room temperature for three days under daylight irradiation.

An LIFDI (FD+) spectrum (in toluene) of a sample of the DMSO solution shows a peak at m/z=890.35 (m/z calculated for [C₃₇H₅₆Pt₂]+: 890.36775) and serves as evidence for the formation of [PtMe₃(Gua-8-Me)-CH₂-(Gua-8-Me)PtMe₃].

### Example 4: Photohydrosilylation of 1-Octene with Pentamethyldisiloxane Using [PtMe₃(GuaMe)] as a Precatalyst

NMR Experiments with 5 ppm of Pt:

### Stock Solution (1.78 M):

500 mg of 1-octene (4.45 mmol), 661 mg of pentamethyldisiloxane (4.45 mmol), 2.5 mL of C₆D₆

Pt complex solution (0.0889 mM=0.0005 mol % compared to stock solution): 0.403 mg of [PtMe₃(GuaMe)] (see Example 2), 5 mL of C₆D₆

0.25 mL of stock solution and 0.25 mL of Pt complex solution were filled into an NMR tube. The tube was shaken and then irradiated with UV light (Osram Ultra Vitalux, 300 W, 220 V, plant lamp) for 5 min. ¹H-NMR spectra were recorded at time 0 h and after about 0.25 h, 0.50 h, 1 h, 2 h, 4 h, 8 h and 24 h.

The invention is not limited to one of the embodiments described above but may be modified in many ways.

It can be seen that the invention relates to a method for producing complexes of precious metals, in particular platinum, which have at least one organo-dihydroazulenyl ligand. The invention also relates to complexes of precious metals, in particular platinum, which have at least one organo-dihydroazulenyl ligand and to the use of the aforementioned metal complexes as precatalysts and/or catalysts in a chemical reaction or as precursor compounds for producing a layer which contains a precious metal, in particular platinum, or a metal layer consisting of a precious metal, in particular platinum, in particular on at least one surface of a substrate. The invention additionally relates to a substrate, in particular a substrate which can be obtained according to such a method. The invention also relates to a crosslinkable silicon composition comprising at least one compound with aliphatic carbon-carbon multi-bonds, at least one compound with Si-bonded hydrogen atoms, and at least one platinum (IV) complex of the aforementioned type. The invention also relates to novel alkali metal organo-dihydroazulenyls which can be used to produce metal complexes of the aforementioned type.

With the method described herein, complexes of precious metals, in particular platinum, can be produced in a high purity and good yield in a simple, reproducible and comparatively cost-effective manner. The method can also be carried out on an industrial scale with a comparable yield and purity of the target compounds. The metal complexes obtainable by means of the method described above represent a relatively cost-effective and particularly sustainable alternative to metal complexes comprising cyclopentadienyl ligands. This applies in particular to use as precatalysts and/or catalysts in chemical reactions for the production of crosslinkable silicone compositions, by means of which the production of silicone elastomers can be realized in a particularly simple, reliable and cost-effective manner. The platinum(IV) complexes, in particular [PtMe₃(GuaMe)], can advantageously be used, for example, as precatalysts and/or catalysts in light-induced platinum-catalyzed hydrosilylation reactions, the hydrogenation of unsaturated compounds, and polymerization reactions in which the activation is effected by ultraviolet or visible radiation. In addition, the platinum(IV) complexes, in particular [PtMe₃(GuaMe)], are particularly suitable as precursor compounds for producing high-quality substrates having at least one platinum-containing layer or at least one platinum layer on at least one surface. Furthermore, the present invention extends the spectrum of alkali metal organo-dihydroguaiazulenides that can be used for the production of metal complexes, in particular of the aforementioned type.

All features and advantages resulting from the claims, the description and the figures, including constructive details, spatial arrangements and method steps, can be essential to the invention, both in themselves and in the most diverse combinations.

1. Compound according to general formula 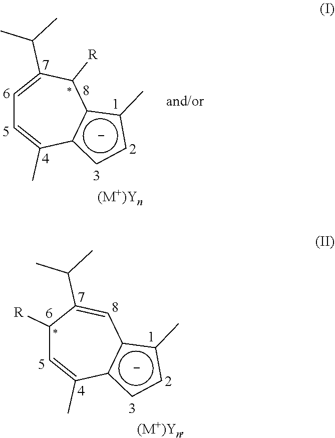
wherein
M⁺ is an alkali metal cation,
R is selected from the group consisting of primary, secondary, tertiary alkyl, alkenyl and alkynyl radicals having 1 to 10 carbon atoms, cyclic alkyl radicals having 3 to 10 carbon atoms, a benzyl radical, mononuclear aryl radicals, polynuclear aryl radicals, mononuclear heteroaryl radicals and polynuclear heteroaryl radicals,
Y is a neutral ligand that is bound or coordinated to M⁺ via at least one donor atom, wherein H₂O is excluded,
and
n=0, 1, 2, 3 or 4,
wherein
the compounds lithium-7-iso-propyl-1,4,8-trimethyl-dihydroazulenide and lithium-7-iso-propyl-1,4-dimethyl-8-phenyl-dihydroazulenide and their THE adducts and DME adducts are excluded.

2. Compound according to claim 1, wherein
i. there is an isomerically pure compound according to general formula I or general formula II
or
ii. there is an isomer mixture containing a first regioisomer according to formula I and a second regioisomer according to formula II.

3. Compound according to claim 2, wherein an isomer ratio of first regioisomer: second regioisomer is ≥80:20 and <90:10.

4. Compound according to claim 1, wherein the alkali metal cation M⁺ is selected from the group consisting of Li⁺, Na⁺ and K⁺.

5. Compound according to claim 1, wherein the neutral ligand Y
A) is a polar aprotic solvent
or
B) is a crown ether
selected from the group consisting of macrocyclic polyethers and aza-, phospha- and thia-derivatives thereof,
wherein an internal diameter of the crown ether and an ion radius of the alkali metal cation M⁺ correspond to each other.

6. Use of a compound according to general formula I and/or according to general formula II according to claim 1 for producing a platinum(IV) complex according to general formula 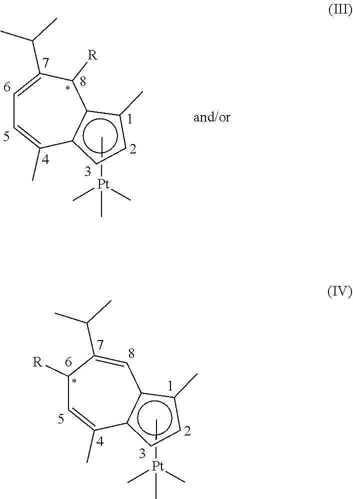
wherein
R is selected from the group consisting of primary, secondary, tertiary alkyl, alkenyl and alkynyl radicals having 1 to 10 carbon atoms, cyclic alkyl radicals having 3 to 10 carbon atoms, a benzyl radical, mononuclear aryl radicals, polynuclear aryl radicals, mononuclear heteroaryl radicals and polynuclear heteroaryl radicals.

7. Method for producing a platinum(IV) complex according to general formula 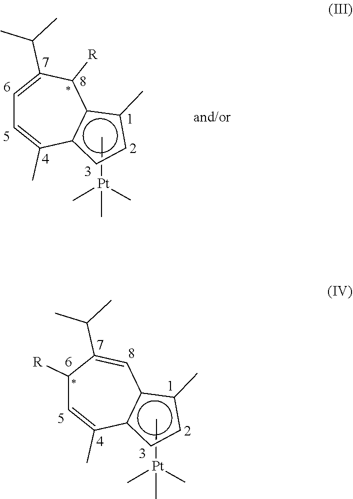
wherein
R is selected from the group consisting of primary, secondary, tertiary alkyl, alkenyl and alkynyl radicals having 1 to 10 carbon atoms, cyclic alkyl radicals having 3 to 10 carbon atoms, a benzyl radical, mononuclear aryl radicals, polynuclear aryl radicals, mononuclear heteroaryl radicals and polynuclear heteroaryl radicals,
using
a compound according to the general formula I and/or general formula II according to claim 1,
comprising the steps of:
A. providing
the compound according to general formula I and/or general formula II
and
a platinum precursor,
B. synthesizing the platinum(IV) complex according to general formula III and/or general formula IV
using the compound according to general formula I and/or general formula II as a reactant
in a solvent S_{P},
C. optionally isolating the platinum(IV) complex synthesized in step B according to general formula III and/or general formula IV.

8. Method according to claim 7, wherein the solvent S_{P} comprises at least one solvent selected from the group consisting of polar aprotic solvents, aliphatic hydrocarbons, aromatic hydrocarbons, organosilicon compounds, and mixtures thereof.

9. Method according to claim 7, wherein the synthesis in step B comprises at least one salt metathesis reaction.

10. Method according to claim 7, wherein the platinum precursor to be provided in step A. is selected from the group consisting of trimethylplatinum(IV) iodide, trimethylplatinum(IV) bromide and trimethylplatinum(IV) chloride, and mixtures thereof, wherein provision takes place
i. as a solution comprising the platinum precursor and a solvent SA
or
ii. as a solid.

11. Platinum(IV) complex according to general formula 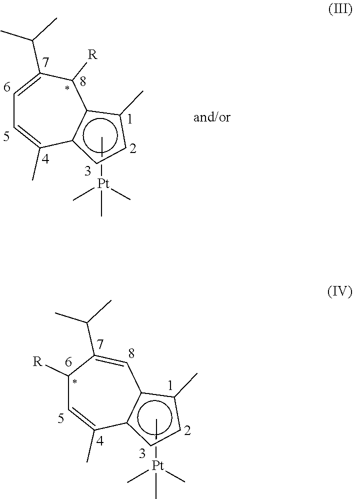
or
solution or suspension comprising a platinum(IV) complex according to general formula 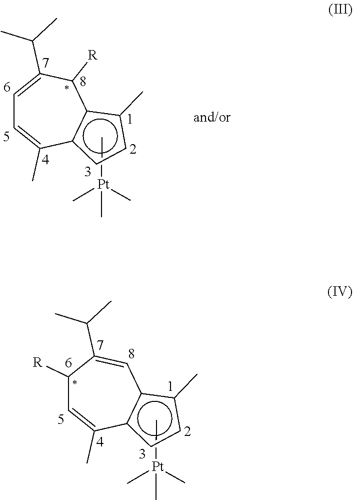
and a solvent that is in particular miscible with or identical to the solvent S_{P},
wherein
R is selected from the group consisting of primary, secondary, tertiary alkyl, alkenyl and alkynyl radicals having 1 to 10 carbon atoms, cyclic alkyl radicals having 3 to 10 carbon atoms, a benzyl radical, mononuclear aryl radicals, polynuclear aryl radicals, mononuclear heteroaryl radicals and polynuclear heteroaryl radicals,
obtained or obtainable according to a method according to claim 7.

12. Platinum(JV) complex according to general formula 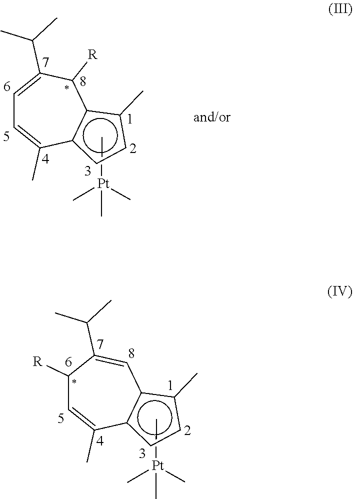
or
solution or suspension comprising a platinum(IV) complex according to general formula 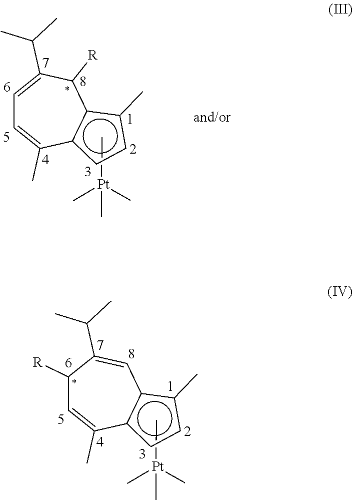
and a solvent,
wherein
R is selected from the group consisting of primary, secondary, tertiary alkyl, alkenyl and alkynyl radicals having 1 to 10 carbon atoms, cyclic alkyl radicals having 3 to 10 carbon atoms, a benzyl radical, mononuclear aryl radicals, polynuclear aryl radicals, mononuclear heteroaryl radicals and polynuclear heteroaryl radicals.

13. Platinum(IV) complex or solution or suspension according to claim 11, wherein there is a diastereomer mixture comprising
a first diastereomer according to formula III.D1 and a second diastereomer according to formula III.D2, 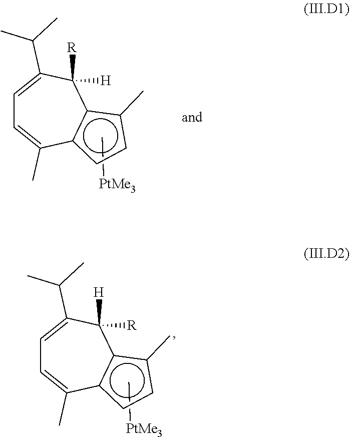
and/or
a third diastereomer according to formula IV.D3 and a fourth diastereomer according to formula IV.D4, 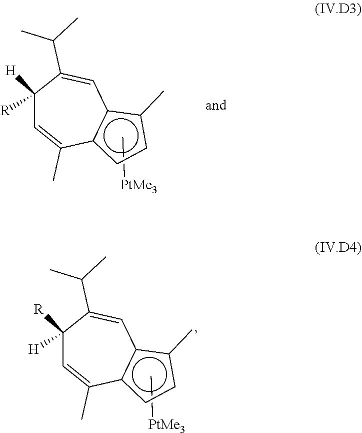
wherein each diastereomer is present as an enantiomer mixture.

14. The platinum(IV) complex or solution or suspension according to claim 11, wherein at least one diastereomer is present as a racemate.

15. Platinum(IV) complex or solution or suspension according to claim 13, wherein there is a mixture of two diastereomers and wherein a diastereomeric ratio of
first diastereomer: second diastereomer
or
third diastereomer: fourth diastereomer
is ≥60:40 and <90:10.

16. Platinum(IV) complex or solution or suspension according to claim 13, wherein there is a diastereomer mixture consisting of the first diastereomer according to formula III.D1 and the second diastereomer according to formula III.D2, 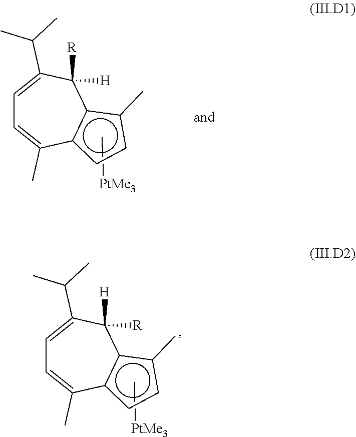
wherein each diastereomer is present as an enantiomer mixture.

17. Platinum(IV) complex according to formula 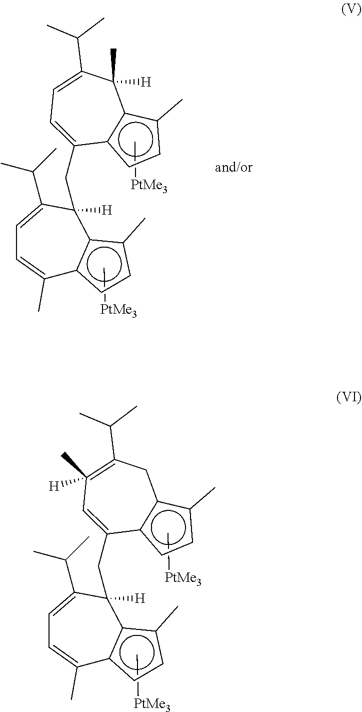
or
solution or suspension comprising a platinum(IV) complex according to formula V and/or formula VI and a solvent SD that comprises or is a nonpolar aprotic or a polar aprotic solvent.

18. Use of at least one platinum(IV) complex according to claim 11, as
i. a precatalyst or a solution or suspension containing a precatalyst in a chemical reaction
and/or
ii. a catalyst or a solution or suspension containing a catalyst in a chemical reaction
and/or
iii. a precursor compound or a solution or suspension containing a precursor compound for producing at least one layer consisting of platinum or at least one layer containing platinum on at least one surface of a substrate.

19. Method for carrying out a chemical reaction using at least one platinum(IV) complex according to claim 11,
comprising the steps of:
A) providing
the at least one platinum(IV) complex according to general formula III and/or IV and/or according to formula V and/or VI
or
the at least one solution or suspension,
comprising a platinum(IV) complex according to general formula III and/or IV and a solvent that is in particular miscible with or identical to the solvent S_{P}, and/or comprising a platinum(IV) complex according to formula V and/or VI and a solvent SD that comprises or is a nonpolar aprotic or a polar aprotic solvent,
and
B) carrying out the chemical reaction using the at least one platinum(IV) complex as a precatalyst and/or as a catalyst.

20. Method for producing
i. at least one layer consisting of platinum
or
ii. at least one layer containing platinum
on at least one surface of a substrate using at least one platinum(IV) complex according to claim 11,
comprising the steps of:
A) providing
the at least one platinum(IV) complex according to general formula III and/or IV
or
the at least one solution or suspension,
comprising a platinum(IV) complex according to general formula III and/or IV and a solvent that is in particular miscible with or identical to the solvent S_{P}
and
B) depositing
i. the at least one layer consisting of platinum
or
ii. the at least one layer containing platinum
on the at least one surface of the substrate using the at least one platinum(IV) complex according to general formula III and/or IV as precursor compound.

21. Substrate comprising
i. at least one layer consisting of platinum
or
ii. at least one layer containing platinum
on at least one surface,
wherein the at least one layer consisting of platinum or the at least one layer containing platinum is producible or produced using
at least one platinum(IV) complex according to claim 11.

22. Crosslinkable silicone composition comprising
i. at least one compound selected from the group consisting of type (a) compounds, type (b) compounds and type (c) compounds, wherein
type (a) compounds are organic compounds and organosilicon compounds, each comprising at least two radicals having aliphatic carbon-carbon multi-bonds,
type (b) compounds are organosilicon compounds, each comprising at least two Si-bonded hydrogen atoms, and
type (c) compounds are organosilicon compounds, each comprising SiC-bonded radicals having aliphatic carbon-carbon multi-bonds and Si-bonded hydrogen atoms,
wherein the silicone composition contains at least one compound having aliphatic carbon-carbon multi-bonds and at least one compound having Si-bonded hydrogen atoms,
and
ii. at least one platinum(IV) complex according to claim 11.

23. Compound according to claim 1, wherein R=Me.
